(19) United States
(12) Patent Application Publication
(10) Pub. No.: US 2026/0207716 A1
(43) Pub. Date: Jul. 23, 2026

(54) NICK RESECTION IN CANCER

(71) Applicant: UNIVERSITY OF MASSACHUSETTS, Westborough, MA (US)

(72) Inventors: Sharon B. CANTOR, Westborough, MA (US); Jenna M. WHALEN, Westborough, MA (US)

(21) Appl. No.: 19/143,071

(22) PCT Filed: Jun. 10, 2024
(86) PCT No.: PCT/US2024/033255

§ 371 Date: Jun. 25, 2025

Related U.S. Application Data
(60) Provisional application No. 63/532,846, filed on Aug. 15, 2023.

(51) Int. Cl. A61K 38/46; A61K 31/7105; A61P 35/00; G01N 33/50

(52) U.S. Cl. CPC A61K 38/465; A61K 31/7105; A61P 35/00; G01N 33/5011; C12N 2310/20; C12N 2740/15043

(57) ABSTRACT

Provided herein are methods and compositions for treating cancer by contacting cancer cells with a CRISPR nickase and a single guide RNA (sgRNA) targeting at least one sequence in the cancer cells.

### CLAIM OF PRIORITY

This application claims the benefit U.S. Provisional Patent Application Ser. No. 63/532,846, filed on Aug. 15, 2023. The entire contents of the foregoing are hereby incorporated by reference.

### FEDERALLY SPONSORED RESEARCH OR DEVELOPMENT

This invention was made with Government support under Grant No. CA254037 awarded by the National Institutes of Health. The Government has certain rights in the invention.

### TECHNICAL FIELD

Provided herein are methods and compositions for treating cancer by contacting cancer cells with a CRISPR nickase and a single guide RNA (sgRNA) targeting at least one sequence in the cancer cells.

### BACKGROUND

Genotoxic cancer therapy inflicts a range of DNA damaging lesions including DNA double-strand breaks (DSBs), single stranded breaks (SSBs), crosslinks, and protein trapped DNA complexes. The damage can be insurmountable and trigger cell death. Yet, the diverse nature of this damage often complicates our understanding of which specific lesion is most detrimental to cancer cells and how they eventually develop resistance. Most often the sensitizing lesion stemming from genotoxic therapy is thought to be DSBs that, if not accurately repaired, drive genomic instability and cell death¹⁻³.

### SUMMARY

Provided herein are methods for the treatment of cancers associated with germline mutations in BRCA1/2 and/or somatic loss of p53-binding protein 1 (53BP1), including solid tumors, and any cancer known to have hyper-resection. In one aspect, provided herein are methods for treating a cancer; the methods comprise administering to a cancer cell a CRISPR nickase and at least one single guide RNA (sgRNA) targeting at least one sequence in the cancer cell.

In some embodiments, the sgRNA targets at least two, three, four, five, 10, 15, 20, or 100 (or more, e.g., at least 100, 200, 500, or 1000 or more) sequences in the cancer cell. In some embodiments, the sgRNA targets AAVS1 or a repeat sequence, optionally a CAG repeat.

In some embodiments, the CRISPR nickase is a CAS9 nickase, optionally an SpCas9 with a D10A or H840A mutation. In some embodiments, the nickase is a Cas9 nickase-exonuclease fusion construct, optionally wherein the exonuclease is a t7 exonuclease.

In some embodiments, the nickase and sgRNA are administered as a nucleic acid. In some embodiments, the nucleic acid is a viral vector comprising a sequence encoding the nickase and optionally encoding the sgRNA.

In some embodiments, the nickase and sgRNA are administered as a ribonucleoprotein complex.

In some embodiments, the cancer is a BRCA1-deficient cancer, e.g., BRCA1-deficient breast (e.g., triple negative breast cancer), ovarian, fallopian tube, primary cancer, pancreatic, melanoma, or prostate cancer, optionally wherein the cancer is a PARPi resistant cancer.

In some embodiments, the cancer is a 53BP1-deficient cancer, e.g., breast cancer, pancreatic cancer, prostate cancer, colorectal cancer, or glioblastoma, optionally wherein the cancer is a PARPi resistant cancer.

Also provided herein are methods for identifying a subject who has cancer for treatment using a method described herein, by contacting cells from the cancer with a CRISPR nickase and a single guide RNA (sgRNA) targeting at least one sequence in the cancer cell, and determining viability of the cells after contact with the nickase and sgRNA as compared to viability of control cells in the absence of the nickase and sgRNA, wherein a decrease in viability after contact with the nickase and sgRNA as compared to viability in the control cells indicates that the subject is likely to respond to treatment with a method described herein (e.g., comprising administering to a cancer cell a CRISPR nickase and at least one single guide RNA (sgRNA) targeting at least one sequence in the cancer cell).

In some embodiments, the methods further comprise administering a treatment comprising as described herein (e.g., comprising administering to a cancer cell a CRISPR nickase and at least one single guide RNA (sgRNA) targeting at least one sequence in the cancer cell) to a subject who has a decrease in viability after contact with the nickase and sgRNA.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

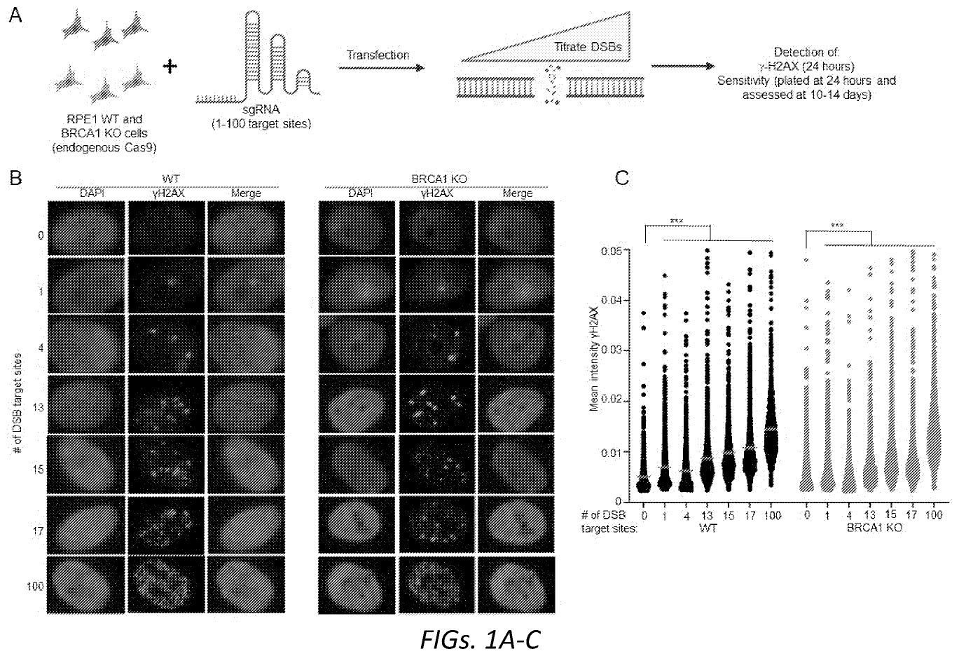

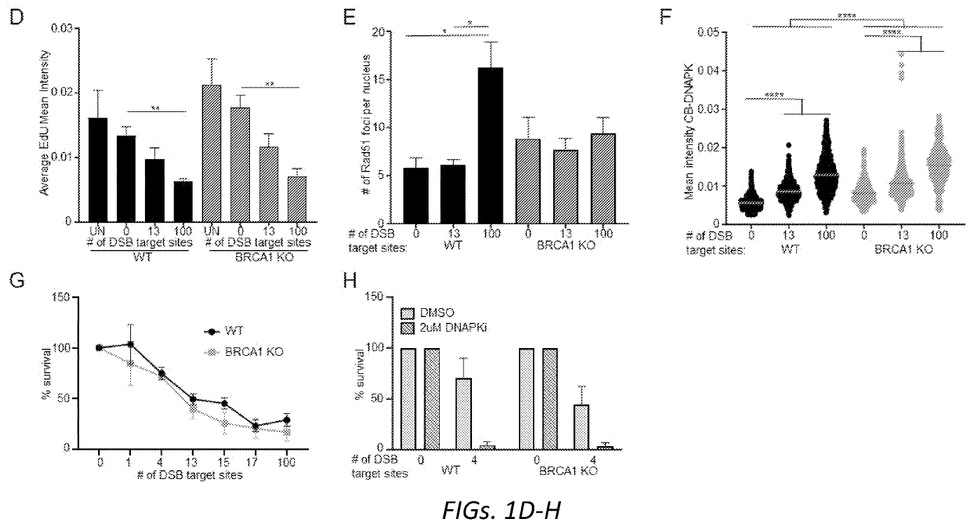

FIGS. 1A-H. BRCA deficient cells are not selectively sensitive to CAS9-induced DSBs
A) Schematic of experimental steps for transfecting sgRNAs by RNAiMax lipofectamine transfection to induce DSBs and assess subsequent γH2AX signal and colony survival.
B) Example images of γH2AX foci (green) and Hoechst staining (blue) in RPE1 wild-type (WT) and BRCA1 KO cell lines with the indicated number of DSBs induced.
C) Quantification of γH2AX foci mean intensity across a range of induced DSBs by Cas9. The mean intensity of immunofluorescence for each nucleus was measured. *** p<0.001 by Student's T-test.
D) 24 hours after Cas9-induced DSBs at 0, 13, or 100 target sites 10 μM EdU was added for 30 minutes followed by Click-It kit. An untreated control (UN) where no transfections buffers were added was included. The mean intensity of each nucleus was measured. ** p<0.01 by Student's T-test.
E) Quantification of Rad51 foci per nucleus 24 hours after Cas9-induced DSBs at 0, 13, or 100 target sites. The number of foci per nucleus was measured. *p<0.05 by Student's T-test.
F) Quantification of CB-DNAPK mean intensity 24 hours after Cas9-induced DSBs at 0, 13, or 100 target sites. The mean intensity of each nucleus was measured. **** p<0.0001 by Student's T-test.
G) Colony cell survival assays for RPE1 WT and BRCA1 KO cells with endogenous Cas9 10-14 days after Cas9-induced DSBs at the indicated number of target sites. Survival was normalized to the 0 DSBs control that did not have the sgRNA added, but was otherwise treated with all the same buffers and conditions. Data represents the mean percentage±SEM of survival for each dot.
H) Colony cell survival assays for RPE1 WT and BRCA1 KO cells with endogenous Cas9 10-14 days after Cas9-induced DSBs at 0 or 4 target sites and treatment with DMSO or 2 μM DNAPKi for the duration of the 10-14 days. Survival was normalized to the 0 DSB control. Data represents the mean percentage±SEM of survival for each bar.

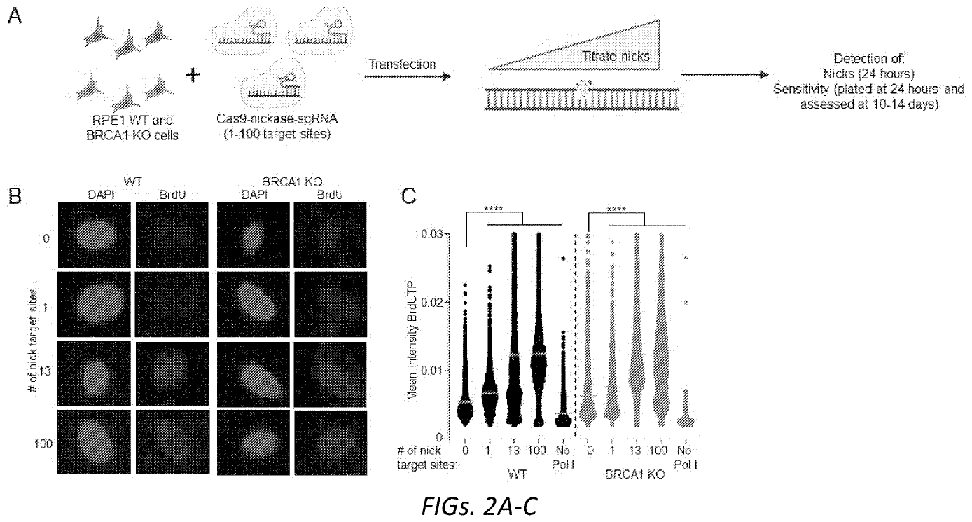

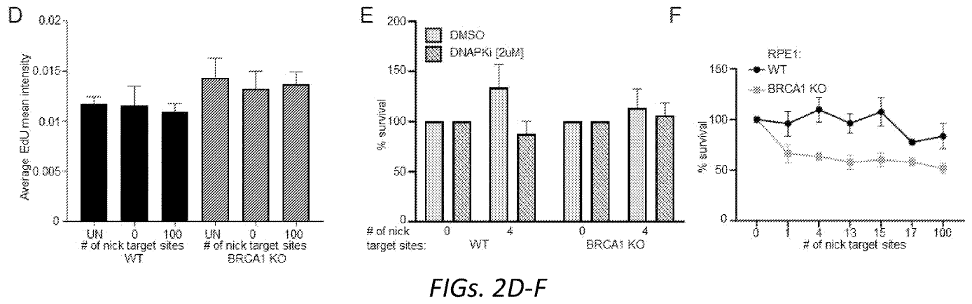

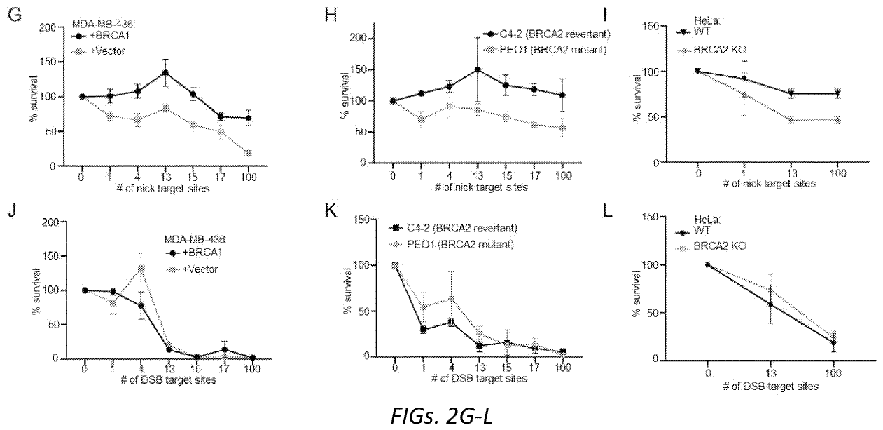

FIGS. 2A-L. BRCA deficient cells are selectively sensitive to Cas9-induced nicks
A) Schematic of experimental steps for transfecting sgRNAs and Cas9-nickase by CRISPR-Max transfection to induce nicks and assess subsequent nicks and sensitivity.
B) Example images of BrdU (nicks) (red) and Hoechst staining (blue) in RPE1 WT and BRCA1 KO cell lines with the indicated number of nicks (D10A) induced.
C) Quantification of BrdU (nicks) mean intensity in RPE1 WT and BRCA1 KO cell lines 24 hours after Cas9-nickase (D10A)-induced nicks at 0, 1,13, or 100 target sites. The mean intensity of each nucleus were measured. A No Pol I condition was used as a control without the polymerase to show low background level of signal. *** p<0.0001 by Student's T-test.
D) 24 hours after Cas9-nickase (D10A)-induced nicks at 0 or 100 target sites, 10 μM EdU was added for 30 minutes followed by Click-It kit. An untreated control (UN) where no transfections buffers were added was included. The mean intensity of each nucleus was measured.
E) Colony cell survival assays for RPE1 WT and BRCA1 KO cells transfected with Cas9-nickase (D10A) 10-14 days after induced nicks at 0 or 4 target sites and treatment with DMSO or 2 uM DNAPKi for the duration of the 10-14 days. Survival was normalized to the 0 nick control. Data represents the mean percentage±SEM of survival for each bar.
F-I) Colony cell survival assays for (F) RPE1, (G) MDA-MB-436, (H) PEO1 and C4-2, and (I) HeLa cells transfected with Cas9-nickase (D10A) and indicated sgRNAs 10-14 days after nick induction. Survival was normalized to the 0 nick control. Data represents the mean percentage±SEM of survival for each dot. Statistical analysis according to paired t-test.
J-L) Colony cell survival assays for (J) MDA-MB-436, (K) PEO1 and C4-2, and (L) HeLa cells transfected with Cas9 and indicated sgRNAs 10-14 days after DSB induction. Survival was normalized to the 0 DSB control. Data represents the mean percentage±SEM of survival for each bar.

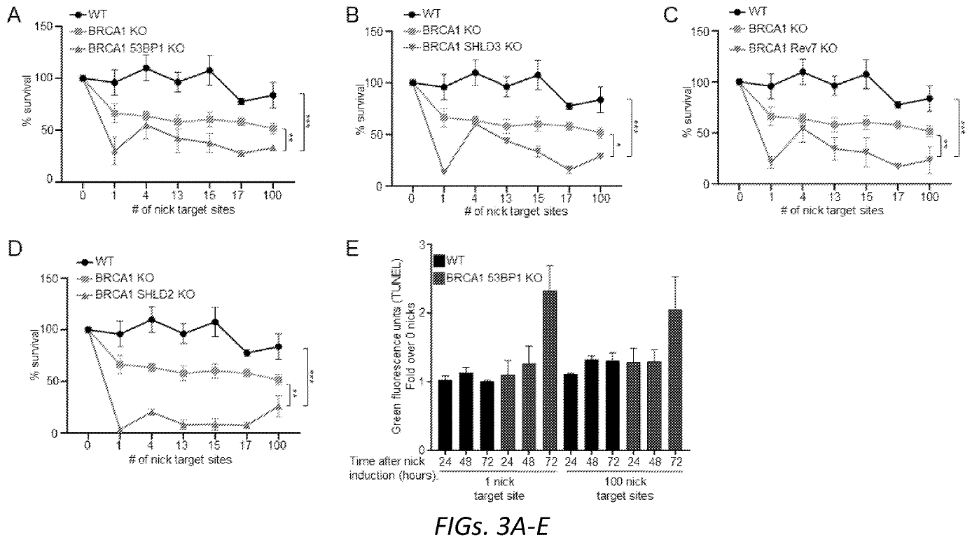

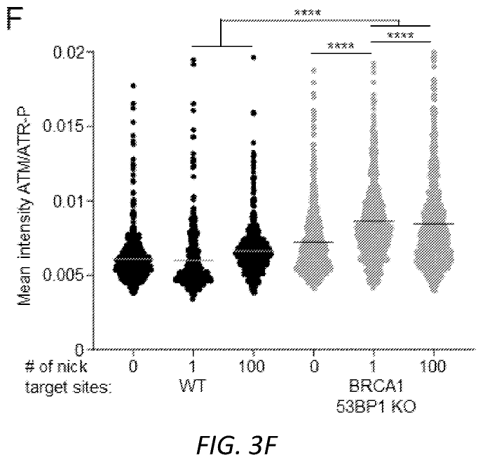

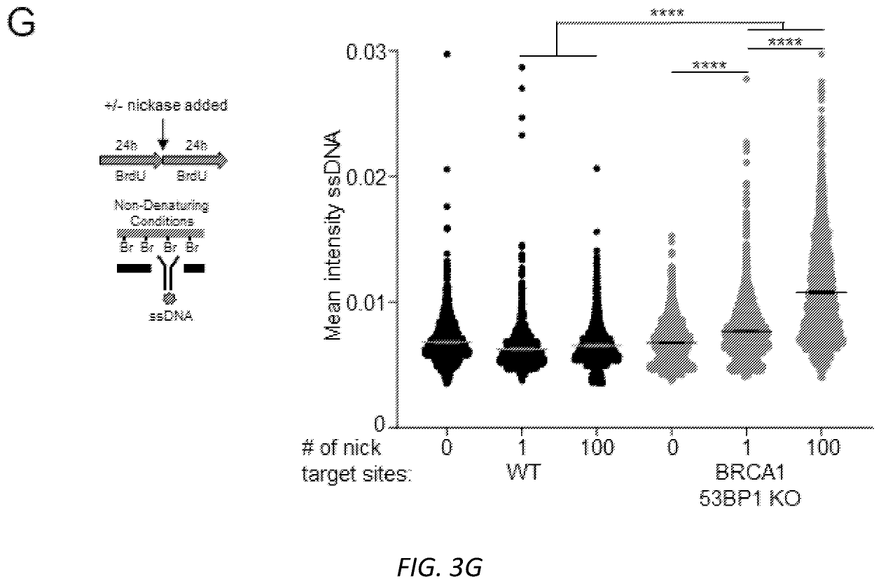

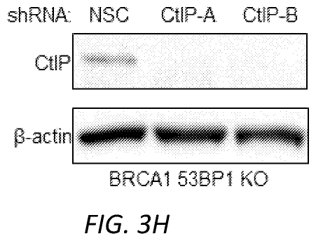

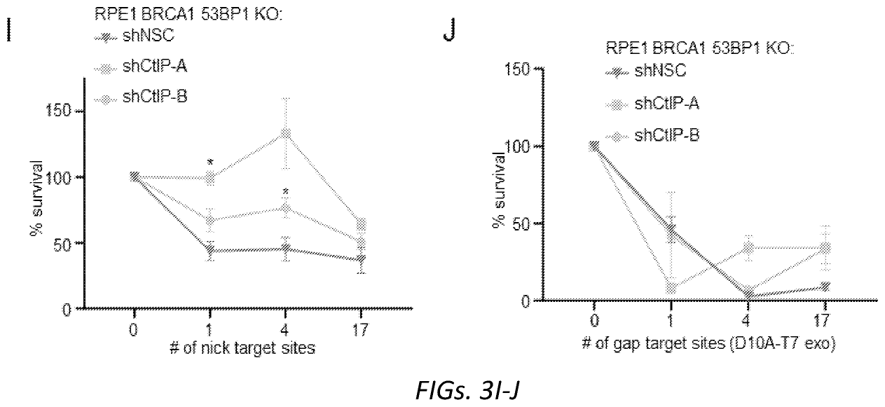

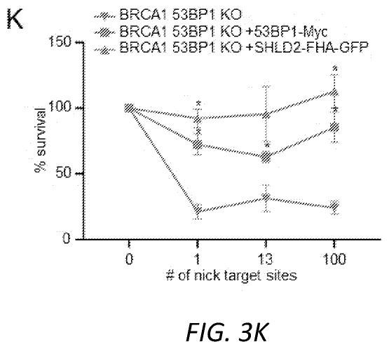

FIGS. 3A-K. Unregulated nick resection generates hyper-sensitivity
A) Colony cell survival assays for RPE1 BRCA1 53BP1 (DKO) cells transfected with Cas9-nickase (D10A) and indicated sgRNAs 10-14 days after nick induction. Survival was normalized to the 0 nick control. Data represents the mean percentage±SEM of survival for each dot. Statistical analysis according to paired t-test.
B-D) Colony cell survival assays for RPE1 (B) BRCA1 SHLD3 KO, (C) BRCA1 Rev7 KO, and (D) BRCA1 SHLD2 KO cells transfected with Cas9-nickase (D10A) and indicated sgRNAs 10-14 days after nick induction. Survival was normalized to the 0 nick control. Data represents the mean percentage±SEM of survival for each dot. Statistical analysis according to paired t-test.
E) Quantification of cell death at 24, 48, and 72 hours after Cas9-nickase (D10A) induced nicks at 0 or 100 target sites in RPE1 WT and DKO cells using a green fluorescent based TUNEL assay. The data was normalized to the 0 nicks condition.
F) Quantification of ATM/ATR phosphorylation mean intensity 24 hours after Cas9-nickase (D10A) induced nicks at 0, 1, or 100 target sites. The mean intensity of immunofluorescence for each nucleus was measured. **** p<0.0001 by Student's T-test.
G) Schematic and quantification of mean ssDNA intensity for RPE1 WT and DKO cells following 24 hours of BrdU pre-labeling and Cas9-nickase (D10A) induced nicks at 0, 1, or 100 target sites. **** p<0.0001 by Student's T-test.
H) Western blot analysis with indicated antibodies of lysates from RPE1 DKO cells expressing small hairpin RNA (shRNA) against non-silencing control (NSC), CtIP (A), and CtIP (B).
I-J) Colony cell survival assays for RPE1 DKO cells after CtIP knockdown with (I) Cas9-nickase (D10A), and (J) Cas9-nickase (D10A) fused to t7 exonuclease, and indicated sgRNAs 10-14 days after nicks were induced at the indicated number of target sites by Cas9-nickase (D10A). Survival was normalized to a 0 nick control. Data represents the mean percentage±SEM of survival for each dot. Statistical analysis according to paired t-test.
K) Colony cell survival assays for RPE1 DKO cells with and without transfection of plasmids containing either 53BP1-Myc or SHLD2-FHA-GFP 10-14 days after Cas9-nickase (D10A) induced nicks at the indicated number of target sites. Survival was normalized to the 0 nick control. Data represents the mean percentage #SEM of survival for each dot. Statistical analysis according to paired t-test.

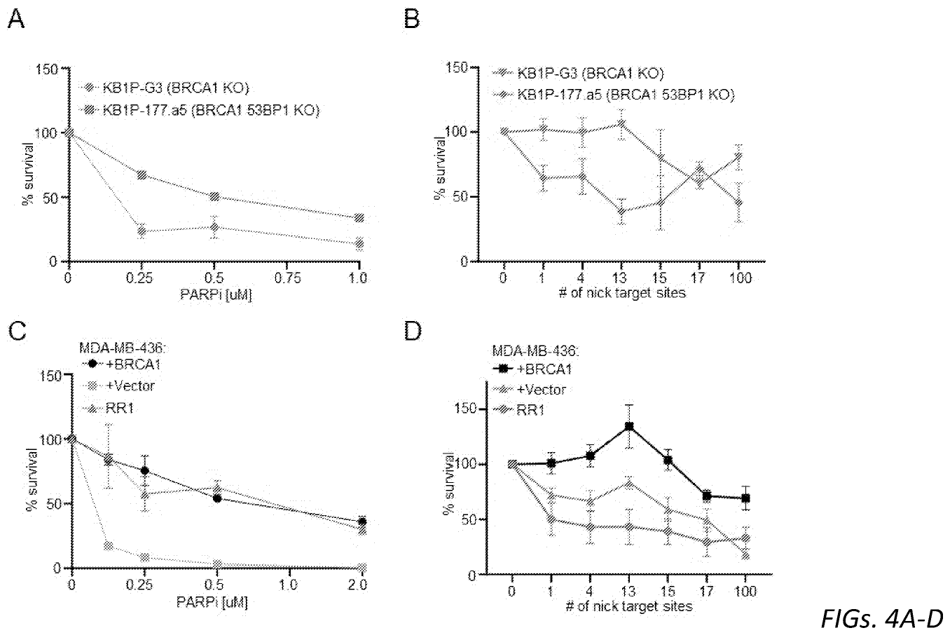

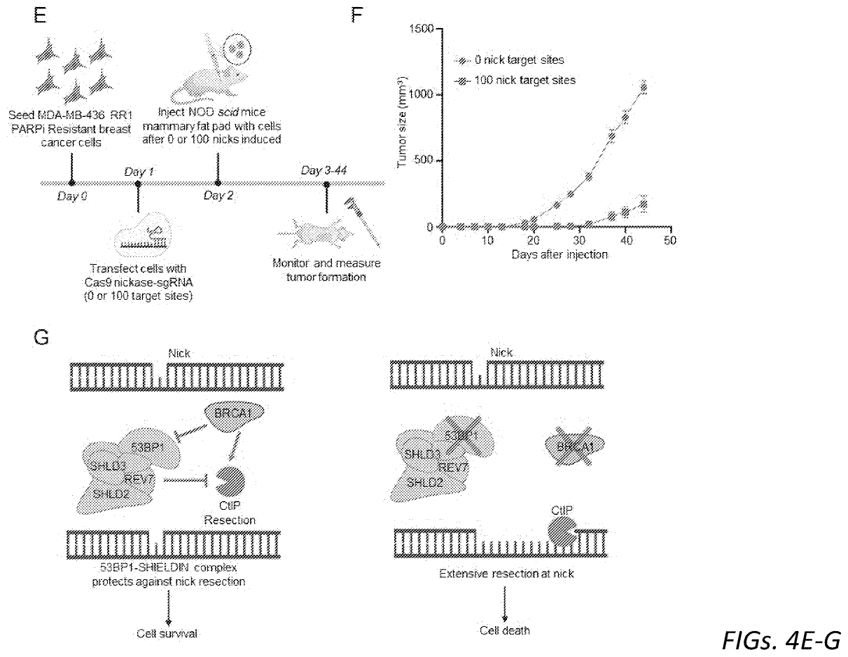

FIGS. 4A-G. Nickases selectively target PARPi resistant BRCA1 mutant cells with restored resection and HR
A-B) Colony cell survival assay for KB1P-G3 (BRCA1 KO) and KB1P-177.a5 (DKO) cells (A) after treatment with the indicated concentrations of PARPi (Olaparib) and (B) after transfection with Cas9-nickase (D10A) and indicated sgRNAs 10-14 days after nicks were induced. Data represents the mean percentage±SEM of survival for each dot. Statistical analysis according to paired t-test.
C-D) Colony cell survival assay for MDA-MB-436 PARPi resistant RR1 vs MDA-MB-436 vector or BRCA1 restored lines (C) after treatment with the indicated concentrations of PARPi (Olaparib) and (D) after transfection with Cas9-nickase (D10A) and indicated sgRNAs 10-14 days after nicks were induced. Data represents the mean percentage±SEM of survival for each dot. Statistical analysis according to paired t-test.
E) Schematic of mouse tumor formation after nick induction.
F) Tumor formation in female NOD scid mice after induction of nicks at 0 or 100 target sites in MDA-MB-436 RR1 cells. Each group contained 6 mice that were injected in the mammary fat pad with 1 million MDA-MB-436 RR1 cells 24 hours after nick induction. Tumor formation and size was monitored for 44 days. Data represents the mean percentage±SEM of tumor size (mm³) in the 6 mice in the group each day measurements were taken. Statistical analysis according to paired t-test.
G) Model showing that in the absence of the 53BP1-Shieldin complex and BRCA1 extensive resection occurs at a nick leading to cell death. This hyper-resection of nicks can be used as a therapy to kill PARPi resistant cancer lacking the 53BP1-Shielin complex.

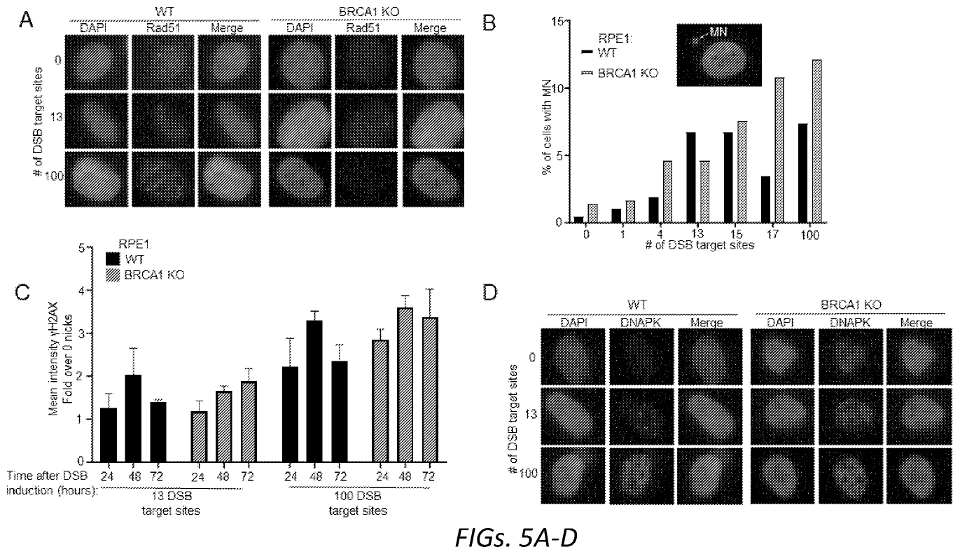

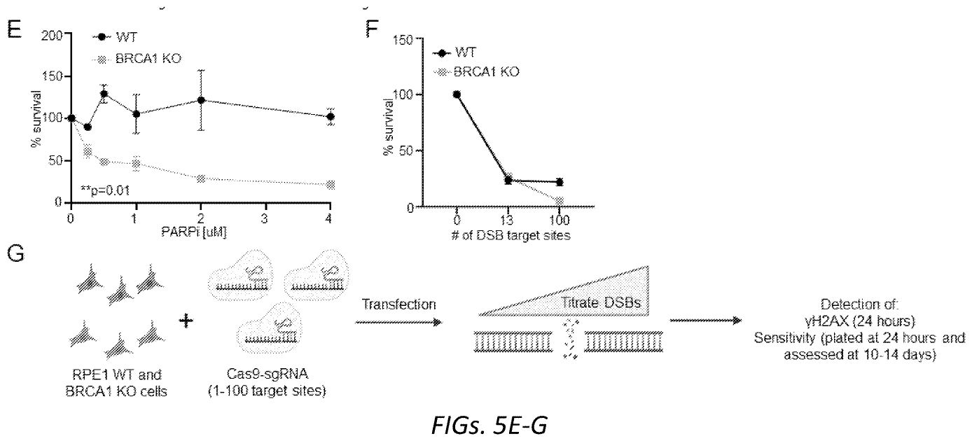

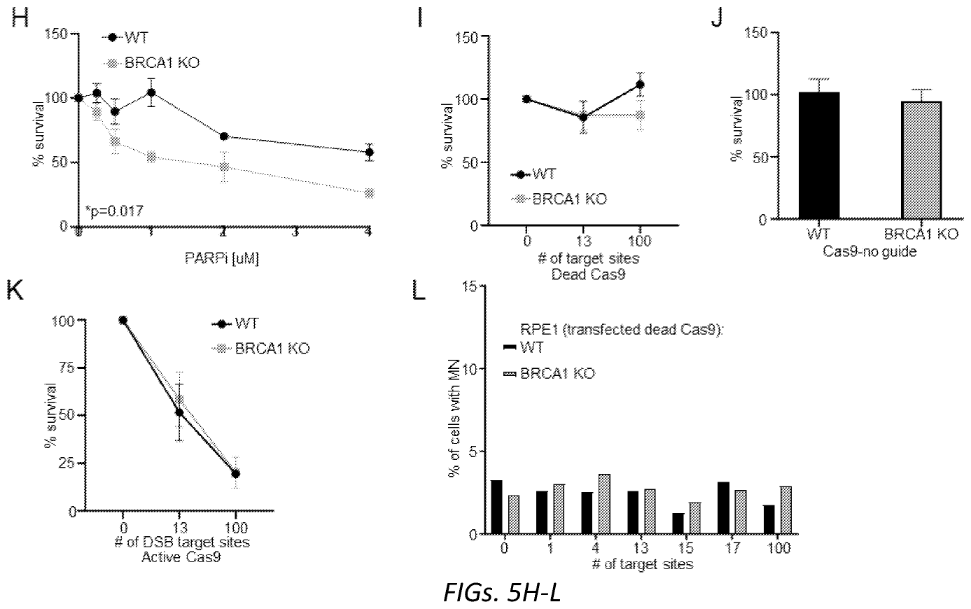

FIGS. 5A-L.
A) Representative images for Rad51 IF (FIG. 1E) after induction of DSBs at the indicated number of target sites by Cas9.
B) The percentage of RPE1 WT and BRCA1 KO cells with micronuclei (MN) was assessed 24 hours after DSB induction using Cas9. The data are representative. The arrow in the example image shows a MN next to the mother nucleus.
C) Quantification of γH2AX mean intensity at 24, 48, and 72 hours after Cas9 induced DSBs at 13 or 100 target sites in RPE1 WT and BRCA1 KO. The data was normalized to the 0 DSBs condition.
D) Representative images for DNAPK IF (FIG. 1F) after induction of DSBs at the indicated number of target sites by Cas9.
E) Colony cell survival assay for RPE1 WT and BCRA1 KO cells (with endogenous Cas9) after treatment with the indicated concentrations of PARPi (Olaparib). Data represents the mean percentage±SEM of survival for each dot. Statistical analysis according to paired t-test.
F) Cell survival assays for RPE1 WT and BRCA1 KO cells (with endogenous Cas9) 5 days after DSB induction by Cas9. Sensitivity was assessed by 96-well format Cell-Titer Glo assays. Survival was normalized to the 0 DSB control. Data represents the mean percentage±SEM of survival for each dot.
G) Schematic of experimental steps for transfecting sgRNAs and Cas9 by CRISPR-Max transfection to induce DSBs and assess subsequent nicks and sensitivity.
H) Colony cell survival assay of RPE1 WT and BRCA1 KO cells (without endogenous Cas9) after treatment with the indicated concentrations of PARPi (Olaparib). Data represents the mean percentage±SEM of survival for each dot. Statistical analysis according to paired t-test.
I-K) Cell survival assays for RPE1 WT and BRCA1 KO cells transfected with (I) catalytically dead Cas9 and sgRNA with targets, (J) active Cas9 and sgRNA without a target, (K) active Cas9 and sgRNA with targets. Survival was normalized to the control that did not have the Cas9 or sgRNA added, but was otherwise treated with all the same buffers and conditions. Data represents the mean percentage±SEM of survival for each bar.
L) The percentage of RPE1 WT and BRCA1 KO cells with micronuclei (MN) was assessed 24 hours after transfection of the catalytically dead Cas9 and sgRNA with the indicated targets.

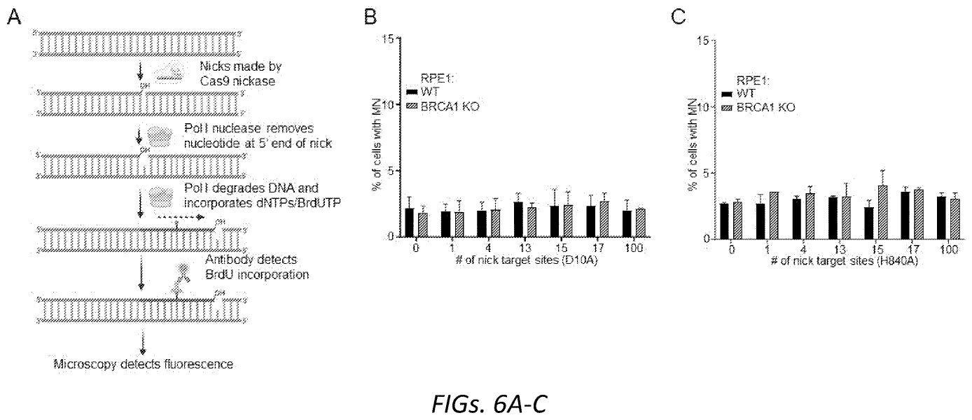

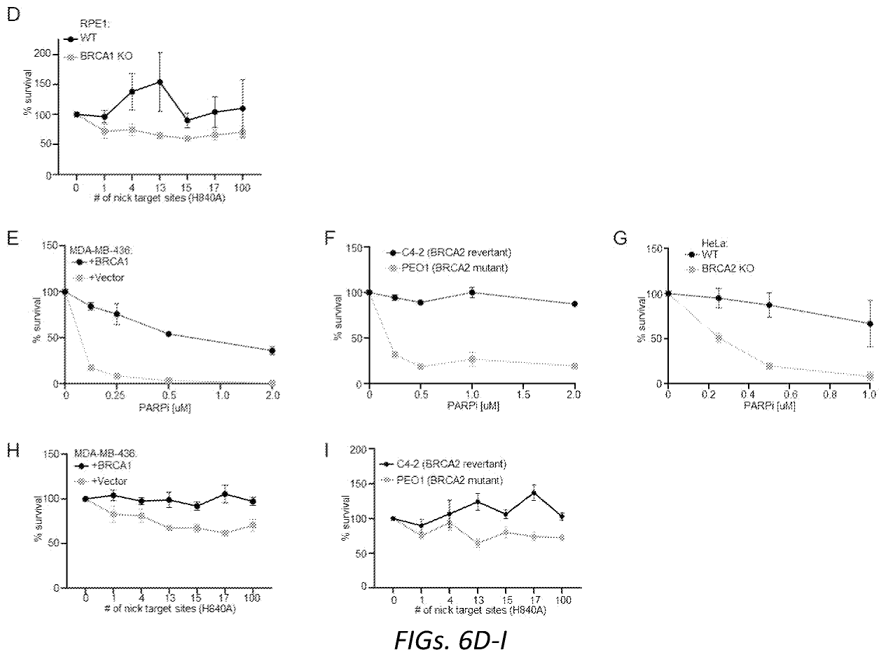

FIGS. 6A-I.
A) Schematic of nick translation assay used to detect the induction of nicks by Cas9-nickase.
B-C) The percentage of RPE1 WT and BRCA1 KO cells with micronuclei (MN) was assessed 24 hours after transfection of (B) Cas9-nickase (D10A) and (C) Cas9-nickase (H840A) and sgRNAs with the indicated targets.
D) Colony cell survival assays for RPE1 WT and BRCA1 KO cells after nick induction by Cas9-nickase (H840A) and indicated sgRNAs. Survival was normalized to the 0 nick control. Data represents the mean percentage±SEM of survival for each dot. Statistical analysis according to paired t-test.
E-G) Colony cell survival assay for (E) MDA-MB-436, (F) PEO1 and C4-2, and (G) HeLa cells after treatment with the indicated concentrations of PARPi (Olaparib). Data represents the mean percentage±SEM of survival for each dot. Statistical analysis according to paired t-test.
H-I) Colony cell survival assays for (H) MDA-MB-436 and (I) PEO1 and C4-2 cells transfected with Cas9-nickase (H840A) and indicated sgRNAs 10-14 days after nick induction. Survival was normalized to the 0 nick control. Data represents the mean percentage±SEM of survival for each dot. Statistical analysis according to paired t-test.

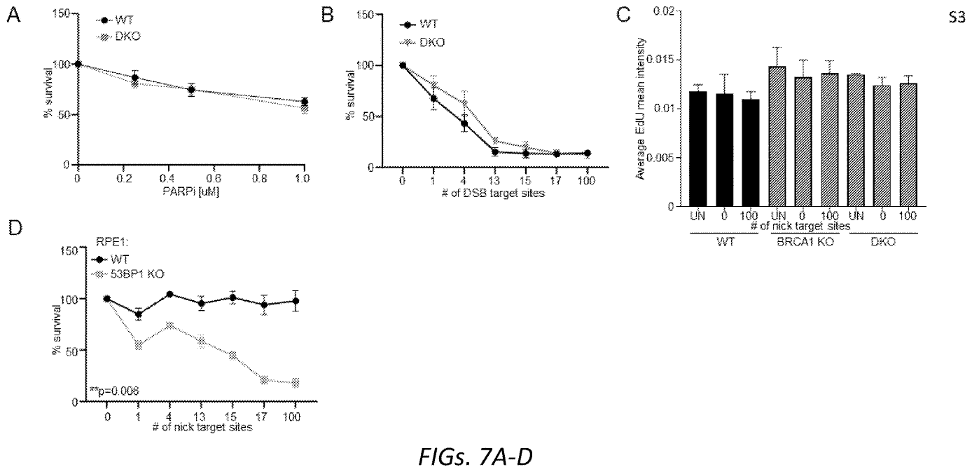

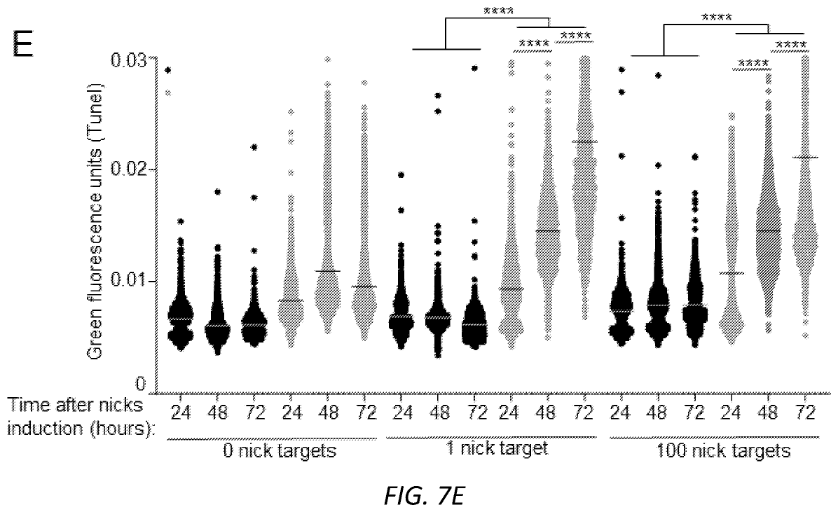

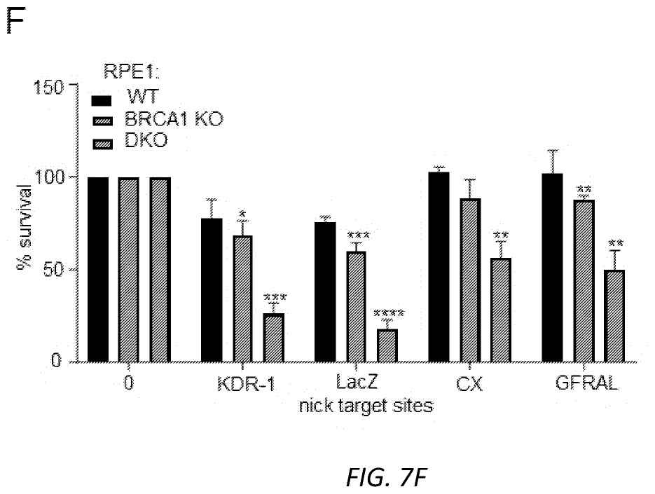

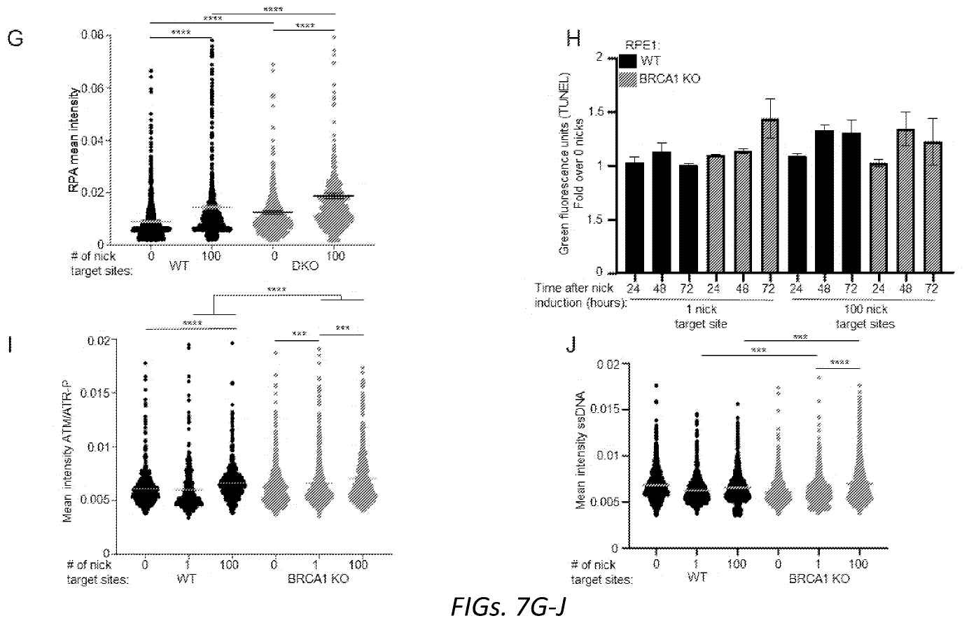

FIGS. 7A-J.
A) Colony cell survival assay for RPE1 WT and DKO cells after treatment with the indicated concentrations of PARPi (Olaparib). Data represents the mean percentage±SEM of survival for each dot.
B) Colony cell survival assays for RPE1 WT and DKO cells transfected with Cas9 and indicated sgRNAs 10-14 days after DSB induction. Survival was normalized to the 0 DSB control. Data represents the mean percentage±SEM of survival for each dot.
C) 24 hours after Cas9-nickase (D10A)-induced nicks at 0 or 100 target sites, 10 μM EdU was added for 30 minutes followed by Click-It kit. An untreated control (UN) where no transfections buffers were added was included. The mean intensity of each nucleus was measured.
D) Colony cell survival assays for RPE1 WT and 53BP1 KO cells transfected with Cas9-nickase (D10A) and indicated sgRNAs 10-14 days after nick induction. Survival was normalized to the 0 nick control. Data represents the mean percentage±SEM of survival for each dot. Statistical analysis according to paired t-test.
E) Quantification of cell death at 24, 48, and 72 hours after Cas9-nickase (D10A)-induced nicks at 0 or 100 target sites in RPE1 WT and BRCA1 KO cells using a green fluorescent based TUNEL assay. **** p<0.0001 by Student's T-test.
F) Colony cell survival assays for RPE1 WT, BRCA1 KO, and DKO cells transfected with Cas9-nickase (D10A) and indicated sgRNAs (each targeting a single site) 10-14 days after nick induction. Survival was normalized to the 0 nick control. Data represents the mean percentage #SEM of survival for each dot. * p<0.05, ** p<0.01, *** p<0.001, and **** p<0.0001 by Student's T-test.
F) Quantification of cell death at 24, 48, and 72 hours after Cas9-nickase (D10A)-induced nicks at 0 or 100 target sites in RPE1 WT and BRCA1 KO cells using a green fluorescent based TUNEL assay. **** p<0.0001 by Student's T-test.
G) Quantification of RPA70 mean intensity 24 hours after Cas9-nickase (D10A)-induced nicks at 0 or 100 target sites. The mean intensity of each nucleus was measured. **** p<0.0001 by Student's T-test.
H) Quantification of cell death at 24, 48, and 72 hours after Cas9-nickase (D10A) induced nicks at 0 or 100 target sites in RPE1 WT and BRCA1 KO cells using a green fluorescent based TUNEL assay. The data was normalized to the 0 nicks condition.
I) Quantification of ATM/ATR phosphorylation mean intensity for RPE1 WT and BRCA1 KO cells 24 hours after Cas9-nickase (D10A) induced nicks at 0, 1, or 100 target sites. The mean intensity of immunofluorescence for each nucleus was measured. *** p<0.001 and **** p<0.0001 by Student's T-test.
J) Quantification of mean ssDNA intensity for RPE1 WT and BRCA1 KO cells following 24 hours of BrdU pre-labeling and Cas9-nickase (D10A) induced nicks at 0, 1, or 100 target sites. *** p<0.001 and **** p<0.0001 by Student's T-test.

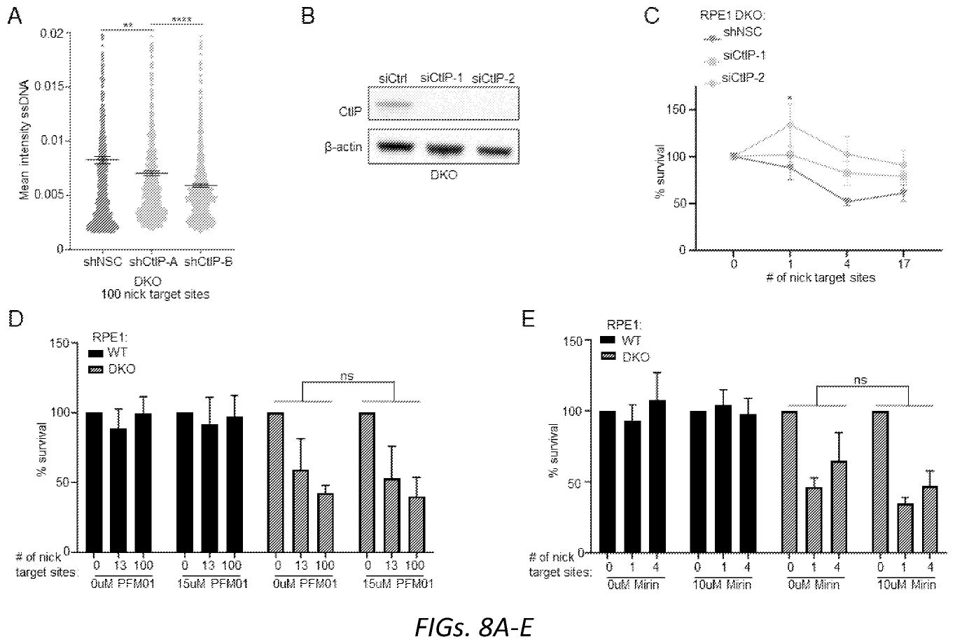

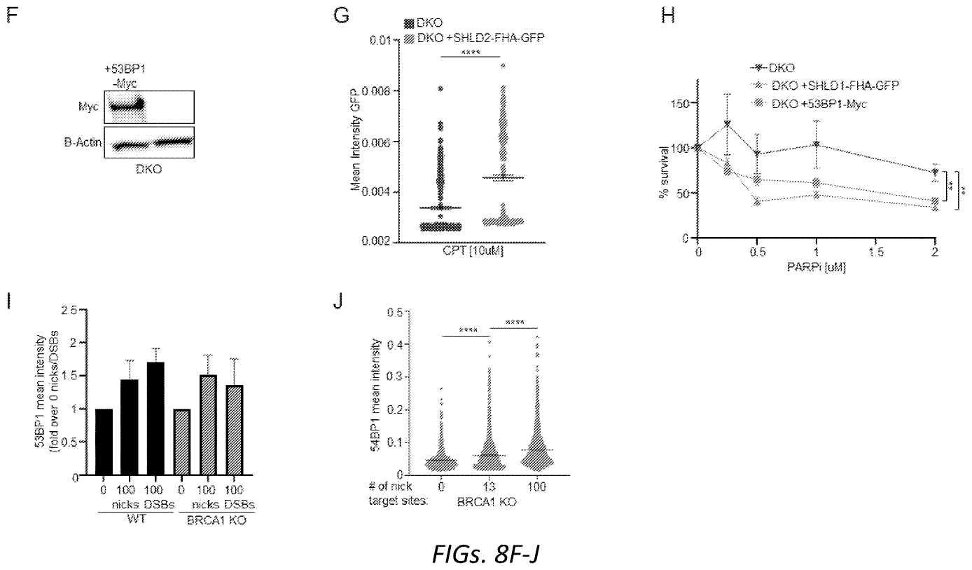

FIGS. 8A-J.
A) Quantification of mean ssDNA intensity for RPE1 DKO cells after CtIP depletion following 24 hours of BrdU pre-labeling and Cas9-nickase (D10A)-induced nicks at 100 target sites. **** p<0.0001 by Student's T-test.
B) Western blot analysis with the indicated antibodies of lysates from RPE1 DKO cells expressing siRNA against non-silencing control and CtIP.
C) Colony cell survival assays for RPE1 DKO cells after CtIP depletion 10-14 days after Cas9-nickase (D10A)-induced nicks at the indicated number of target sites. Survival was normalized to the 0 nick control. Data represents the mean percentage #SEM of survival for each dot. Statistical analysis according to paired t-test.
D-E) Colony cell survival assays for RPE1 WT and DKO cells 10-14 days after Cas9-nickase (D10A)-induced nicks at D) 0, 13, or 100 target sites and treatment with DMSO or 15 μM PFM01 and E) 0, 1, and 4 target sites and treatment with DMSO or 10 μM Mirin for the duration of the 10-14 days. Survival was normalized to the 0 DSB control. Data represents the mean percentage±SEM of survival for each bar. Statistical analysis according to a paired t-test.
F) Western blot analysis with indicated antibodies of lysates from RPE1 DKO cells transfected with a plasmid containing 53BP1-Myc.
G) Quantification of GFP mean fluorescence after treatment with CPT [10 uM] for 1 hour in RPE1 BRCA1 53BP1 KO cells with and without transfection of a plasmid containing SHLD2-FHA-GFP.
H) Colony cell survival assay for RPE1 DKO cells with and without +53BP1-Myc and +SHLD2-FHA-GFP after treatment with the indicated concentrations of PARPi (Olaparib). Data represent the mean percentage±SEM of survival for each dot. Statistical analysis according to paired t-test.
I) Quantification of 53BP1 mean intensity in RPE1 WT and BRCA1 KO cells 24 hours after Cas9 induced DSBs or nicks (D10A) at 0 or 100 target sites. The data was normalized to the 0 nicks condition.
J) Quantification of 53BP1 mean intensity in RPE1 BRCA1 KO cells 24 hours after Cas9-nickase (D10A) induced nicks at 0, 13, or 100 target sites. The mean intensity of immunofluorescence for each nucleus was measured. **** p<0.0001 by Student's T-test.

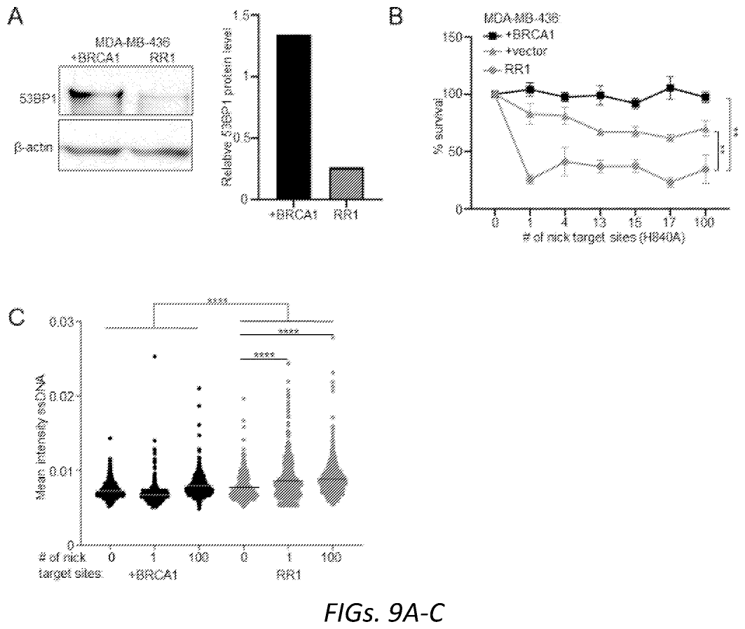

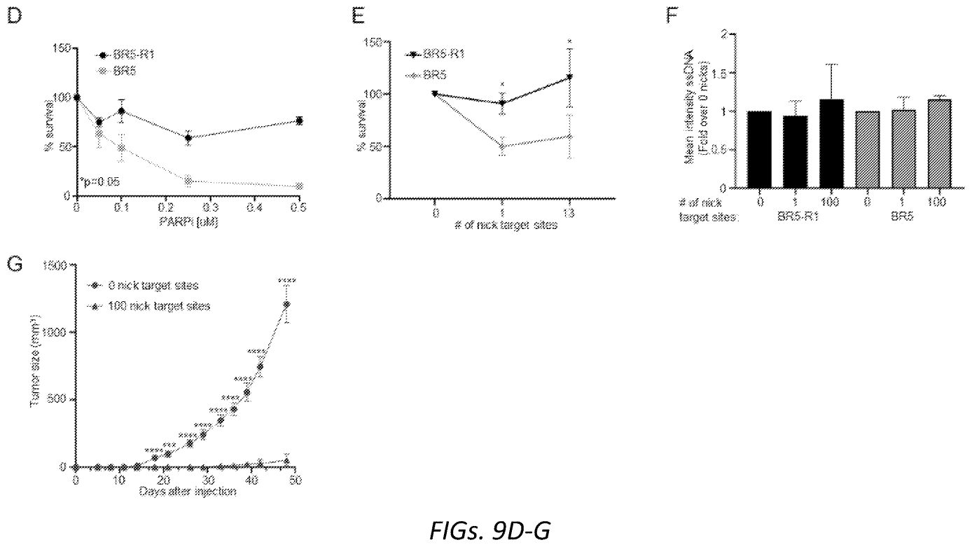

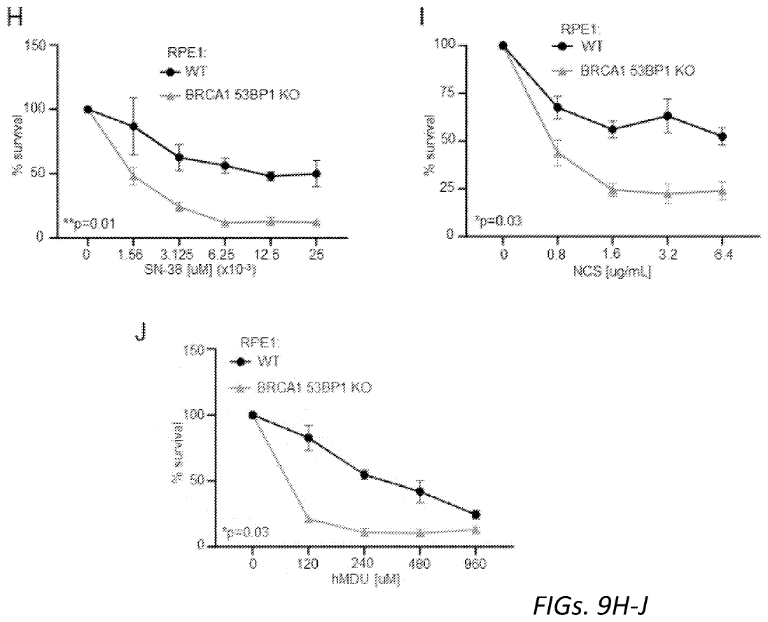

FIGS. 9A-J.
A) Western blot analysis with the indicated antibodies of lysates from MDA-MB-436 cells. Graph shows relative 53BP1 protein levels.
B) Colony cell survival assays for MDA-MB-436 cells with transfected Cas9-nickase (H840A) and indicated sgRNAs 10-14 days after nicks were induced. Survival was normalized to the control that did not have the sgRNA or Cas9-nickase added, but was otherwise treated with all the same buffers and conditions. Data represent the mean percentage±SEM of survival for each dot. Statistical analysis according to paired t-test.
C) Quantification of mean ssDNA intensity for indicated MDA-MB-436 cells following BrdU pre-labeling and nick induction using Cas9-nicase (D10A) at the indicated number of target sites. **** p<0.0001 by Student's T-test.
D-E) Colony cell survival assay for BR5 and BR5-R1 cells (D) after treatment with the indicated concentrations of PARPi (Olaparib) and (E) after transfection with Cas9-nickase (D10A) and indicated sgRNAs 10-14 days after nicks were induced. Data represents the mean percentage±SEM of survival for each dot. Statistical analysis according to paired t-test.
F) Quantification of mean ssDNA intensity for BR5 and BR5-R1 cells following 24 hours of BrdU pre-labeling and 24 hours after Cas9-nickase (D10A)-induced nicks at 0, 1, or 100 target sites. The data was normalized to the 0 nicks condition.
G) Second replicate for FIG. 4F. Tumor formation in female NOD scid mice after induction of nicks at 0 or 100 target sites in MDA-MB-436 RR1 cells. Each group contained 6 mice that were injected in the mammary fat pad with 1 million MDA-MB-436 RR1 cells 24 hours after nick induction. Tumor formation and size was monitored for 48 days. Data represents the mean percentage±SEM of tumor size (mm³) in the 6 mice in the group each day measurements were taken. Statistical analysis according to paired t-test.
H-J) Cell survival assays for RPE1 WT and DKO cells 5 days after addition of the drug H) SN-38, I) neocarzinostatin (NCS), and J) 5-(Hydroxymethyl)-2′-deoxyuridine (hMDU). Sensitivity was assessed by 96-well format Cell-Titer Glo assays. Survival was normalized to the 0 DSB control. Data represents the mean percentage±SEM of survival for each dot.

### DETAILED DESCRIPTION

Extreme sensitivity to genotoxic chemotherapy characterizes cells deficient in the BRCA1 and BRCA2 hereditary breast cancer genes⁴⁻⁶ as they have key functions in the repair of DSBs by homologous recombination (HR)^{7,8}. Thus, targeting the DSB repair defect in BRCA cancers was the impetus for drugs such as Poly (ADP ribose) polymerase 1/2 inhibitors (PARPi)^{9,10} and biomarkers of HR deficiency (HRD) were employed to identify responding tumors¹¹. Challenging the efficacy of precision medicine, PARPi resistance evolves. In BRCA1-deficient tumors, PARPi resistance is achieved by loss of the 53BP1-Shieldin complex that is known for restricting DNA end resection at DSBs¹²⁻¹⁷. The loss of the 53BP1-Shieldin complex reinstates DSB end resection and HR; however, this adaptation exposes vulnerabilities that provides a critical target for therapeutic intervention.

Beyond their role in DSB repair, the BRCA proteins prevent single stranded DNA (ssDNA) replication gaps by supporting lagging strand synthesis, and interestingly, PARP1's involvement in this process means that PARPi can disrupt it¹⁸⁻²⁰. This suggests BRCA-deficient cells' vulnerability might stem from lagging strand anomalies²¹. Indeed, in addition to HR, lagging strand synthesis is revived upon loss of 53BP1, suggesting this restoration might confer PARPi resistance in BRCA1-deficient cells¹⁸. However, ssDNA gaps might also become DSBs, exacerbating issues in BRCA-deficient cells due to defective HR and/or unregulated error-prone non-homologous end joining (NHEJ)²²⁻³⁰. Here, we utilized genetic engineering techniques to show that DNA nicks are uniquely detrimental to BRCA-deficient cells. While gains in DNA end resection reduce PARPi sensitivity as expected, nick sensitivity increases in a resection dependent manner. Together these findings highlight that drugs inducing nicks offer a targeted treatment strategy for refractory tumors that could encompass a wide spectrum of difficult to treat cancers such as triple-negative breast cancer with reduced 53BP1³¹⁻³³, and that limiting nick expansion is a critical function of the 53BP1-Shieldin complex.

A critical finding described herein is that BRCA1 mutant cells that gain PARPi resistance due to loss of the anti-resection 53BP1-Shieldin complex are selectively sensitive to nicks (FIG. 4G). Thus, this anti-resection complex suppresses the excision of DNA at DSB ends⁵⁷ as well as at nicks consistent with the role of 53BP1 in limiting the resection of under-replicated regions to maintain chromatin integrity^{58,59}. We further show the clinical relevance of this nick expansion vulnerability. PARPi resistant 53BP1 low-expressing BRCA1 mutant breast cancer cells are highly sensitive to nicks and when nicks are delivered prior to implantation into a mouse fat pad, tumor formation is largely blocked. Our findings also provide clarity for other BRCA-related toxicity models. For one, given that cells with restored resection and HR are highly sensitive to nicks, it is unlikely that a nick converts to a DSB²²⁻²⁵, but rather that a nick converts to a toxic ssDNA gap. We further identify that DSBs are lethal irrespective of BRCA status when NHEJ is defective. Thus, the non-faithful repair of DSBs by NHEJ is unlikely the cause of toxicity in BRCA-deficient cells²⁸⁻³⁰, but rather that NHEJ counters replication fork recovery⁶⁰. Finally, extensive nick resection induces checkpoints, genomic instability, and cell death independent of replication⁶¹⁻⁶⁴. However, when strand invasion is available, nick resection is limited²⁶. Thus, we speculate that in addition to improved lagging strand synthesis¹⁸, BRCA1 and 53BP1-deficient cells are PARPi resistant because stand invasion curtails resection of lagging strands gaps and promotes their repair. When strand invasion is not available, as in post-replication gaps caused by POLθ inhibitor (POLθi), BRCA1 53BP1-deficient cells are sensitive⁶⁵⁻⁶⁸. An unregulated nick expansion also provides insight for the sensitivity of 53BP1 and BRCA1-deficient cells to ionizing radiation that induces SSBs⁶⁹⁻⁷³; why resection stratifies BRCA mutant cells for sensitivity to POLθ inhibitors (POLθi) 74; and for why depletion of pro-resection factors such as CtiP elevates POLθ inhibitor resistance^{65,66} In summary, our findings highlight that a critical function of the 53BP1-Shieldin complex is to counter nick expansion (FIG. 4G). Therefore, inducing nicks is a promising tool for pro-nick resection cancers that resemble BRCA1 deficiency with reduced 53BP1 including triple-negative breast cancer as well as BRCA proficient but 53BP1 mutated cancers^{31-33,75-83}, such as breast cancer, pancreatic cancer, prostate cancer, colorectal cancer, and glioblastoma.

Thus, provided herein are methods for identifying and treating cancers based on nick status.

### Methods of Treatment

The methods described herein include methods for the treatment of cancers associated with germline mutations in BRCA1/2 and/or somatic loss of p53-binding protein 1 (53BP1), including solid tumors, and any cancer known to have hyper-resection. In some embodiments, the disorder is breast (e.g., triple negative breast cancer), ovarian, fallopian tube, primary cancer, pancreatic, melanoma, prostate cancer, colorectal cancer, or glioblastoma. See, e.g., Petrucelli et al., “BRCA1- and BRCA2-Associated Hereditary Breast and Ovarian Cancer.” 1998 Sep. 4 [Updated 2023 Sep. 21]. In: Adam M P, Feldman J, Mirzaa G M, et al., editors. GeneReviews® [Internet]. Seattle (WA): University of Washington, Seattle; 1993-2024. Available from: ncbi.nlm.nih.gov/books/NBK1247/. In addition, the present methods provide a way to re-sensitize previously PARPi resistant cancers (DKO).

Generally, the methods include administering a therapeutically effective amount of a treatment as described herein, to a subject who is in need of, or who has been determined to be in need of, such treatment. In some embodiments, the methods include administering a therapeutically effective amount of a treatment comprising a Cas nickase and gRNA, alone or in combination with a standard treatment comprising chemotherapy, radiotherapy, and/or resection.

As used in this context, to “treat” means to ameliorate at least one symptom of the disorder associated with abnormal apoptotic or differentiative processes. For example, a treatment can result in a reduction in tumor size or growth rate. Administration of a therapeutically effective amount of a compound described herein for the treatment of a condition associated with abnormal apoptotic or differentiative processes will result in a reduction in tumor size or decreased growth rate, a reduction in risk or frequency of reoccurrence, a delay in reoccurrence, a reduction in metastasis, increased survival, and/or decreased morbidity and mortality, inter alia.

Methods for identifying subjects as having a cancer associated with germline mutations in BRCA1/2 and/or somatic loss of 53BP1 are known in the art and include identification of a heterozygous germline pathogenic (or likely pathogenic) variant in BRCA1 or BRCA2, or a somatic loss-of-function mutation in 53BP1 in the cancer cells, based on molecular genetic testing (see, e.g., Petrucelli et al., 1998, supra; Zhang et al., Cancer Lett. 2021 Mar. 31:501:43-54).

### Cas Nickases

The present methods can use any nickase, including Cas9 and Cas12 nickases. Table A provides a list of exemplary Cas9 and Cas12a orthologs with their nickase mutations.

TABLE A
List of Exemplary Cas9 and Cas12a Orthologs (see WO2018218166 for references)

| | UniProt or | |
|---|---|---|
| | GenBank | |
| | Accession | |
| Ortholog | Number | Nickase Mutations/Catalytic residues* |
| S. pyogenes Cas9 | Q99ZW2.1 | D10A (RuvC), E762A (RuvC), D839A |
| (SpCas9) | | (HNH), H840A (HNH), N854A (HNH |
| | | partial), N863A (HNH), D983A (RuvC), |
| | | D986A (RuvC) |
| S. aureus Cas9 (SaCas9) | J7RUA5.1 | D10A (RuvC), D556A (HNH), H557A |
| | | (HNH), N571A (HNH), N580 (HNH) |
| S. thermophilus Cas9 | G3ECR1.2 | D31A (RuvC), D867A (HNH), H868A |
| (St1Cas9) | | (HNH), N882A, (HNH), N891A (HNH) |
| N. meningitidis Cas9 | CBA04028.1 | D16A (RuvC), D587A (HNH), H588A |
| (Nme1sCas9) | | (HNH), N602A (HNH), N611A (HNH) |
| N. meningitidis DE10444 | WP_002230835.1 | D16A (RuvC), D587A (HNH), H588A |
| Cas9 (Nme2Cas9) | | (HNH), N602A (HNH), N611A (HNH) |
| S. pasteurianus Cas9 | BAK30384.1 | D10A (RuvC), D598A (HNH), H599A |
| (SpaCas9) | | (HNH), N613A (HNH), N622A (HNH) |
| C. jejuni Cas9 (CjCas9) | Q0P897.1 | D8A (RuvC), D558A (HNH), H559A |
| | | (HNH), N573A (HNH), N582A (HNH) |
| F. novicida Cas9 | A0Q5Y3.1 | D11A (RuvC), D968A (HNH), H969A |
| (FnCas9) | | (HNH), N986A (HNH), N995A (HNH) |
| P. lavamentivorans Cas9 | A7HP89.1 | D8A (RuvC), D600A (HNH), H601A |
| (PlCas9) | | (HNH), N615A (HNH), N624A (HNH) |
| C. lari Cas9 (ClCas9) | G1UFN3.1 | D7A (RuvC), D566A (HNH), H567 (HNH) |
| Pasteurella multocida | Q9CLT2.1 | D13A (RuvC), D579A (HNH), H580A |
| Cas9 | | (HNH), |
| F. novicida Cpf1 | A0Q7Q2.1 | D917A, E1006A, D1255A (all RuvC) |
| (FnCpf1) | | |
| M. bovoculi Cpf1 | WP_052585281.1 | D986A (RuvC) |
| (MbCpf1) | | |
| A. sp. BV3L6 Cpf1 | U2UMQ6.1 | D90A8, E993A, Q1226A, D1263A (all |
| (AsCpf1) | | RuvC) |
| L. bacterium N2006 | A0A182DWE3.1 | D832A (RuvC) |
| (LbCpf1) | | |

Variants of the above can also be used, including those with mutations that have improved on-target activity (or even increased off-target activity) or altered PAM requirements, including S. pyogenes Cas9 (SpCas9) variants eSpCas9; HF1; HypaCas9; evoCas9; HiFi Cas9; Sniper-Cas9; Blackjack; LZ3; SuperFi-Cas9; Sniper2L; Sniper2P; rCas9HF; xCas9; SpCas9-NG; VQR/VRER; SpCas9-VRQR; S. aureus Cas9 (SaCas9)-KKH; SpCas9-VRQR; SpRY; SpCas9-NG; SpCas9-QQR; iCas9; SpCas9-L1206P enAsCas12a; enAsCas12a-HF; enLbCas12a (HF); enFnCas12a (HF); chimeric Cas9; cCas9; Streptococcus macacae (Smac) Cas9 NCTC 11558; Spy-mac Cas9, Smac-py Cas9, iSpyMac; N. meningitidis Nme2Cas9; SpG Cas9; SpRY Cas9; SpCas9-NRCH; SpCas9-NRRH; SpCas9-NRTH; or variants described in PCT/US2021/014933 or in Spencer and Zhang, Sci Rep. 2017 Dec. 4; 7(1):16836.

In some embodiments, the nickase is present in a fusion protein also comprising an exonuclease, e.g., a t7, 15, Mre11, lambda, ExoVIII, RecJ, Exo1, or ExoII nuclease.

### Guide RNAs

Each nickase uses a corresponding guide RNA that includes spacer sequences to direct the nickase to target sequences on the genomic DNA; sequences and structures for the guide RNAs are known in the art; where available, single guide RNA (sgRNA) are preferred. In the present methods and compositions, one or more guide RNAs that direct the nickase to one or multiple target sequences can be used. In some embodiments, the guide RNA targets one or more places in the genome; for example, the guide RNA can target repetitive sequences e.g., CAG repeats, that appear multiple times in the genome. Alternatively, a pool of guide RNAs can be used the direct the nickase to multiple target sequences in the genome. Exemplary target sequences include the KDR, LACZ, CXOrf66, and GRFAL genes, which have been identified as nonessential genes. In the present methods and compositions, the gRNA preferably does not target BRCA1/2 or 53BP1.

### Delivery

The nickase and guide RNA can be delivered to the cancer as nucleic acids, e.g., nucleic acids encoding both the nickase and gRNA, or a nucleic acid (e.g., an mRNA) encoding only the nickase, and the gRNA delivered as RNA; viral vectors such as lentivirus, adenovirus, adeno-associated virus, or oncolytic viruses such as herpes simplex virus (HSV) can be used (where the sequences encoding the nickase and optionally gRNA can be delivered in an expression construct comprising a promoter that drives expression of the nickase and gRNA), as can mRNA. Alternatively, the nickase and guide RNA are delivered as ribonucleoprotein (RNP) complexes. The RNPs can be delivered, e.g., in extracellular vesicles (EVs) or lipid nanoparticles or other nanocomplexes such as inorganic nanoparticles. See, e.g., Ahmadi et al., Cancer Gene Therapy 30:936-954 (2023); Song et al., Adv Drug Deliv Rev. 2021 January: 168: 158-180; Bukhari et al., Nano. 2021; 16:2130011: Rabaan et al., Curr Oncol. 2023 February; 30(2):1954-1976.

Preferably the nickase and gRNA are delivered locally to the cancer, e.g., by direct injection into or near a tumor, or administration to the tumor site during surgical resection, but systemic delivery can also be used, e.g., by intravenous or subcutaneous injection.

### EXAMPLES

The invention is further described in the following examples, which do not limit the scope of the invention described in the claims.

### Materials and Methods

The following materials and methods were used in the Examples below unless otherwise indicated.

Cell Lines: Human RPE1-hTERT, PEO1, C4-2, and HeLa cell lines were grown in DMEM supplemented with 10% FBS and 1% Pen Strep (100 U/ml). MDA-MB-436 cell lines were grown in RPMI supplemented with 10% FBS and 1% Pen Strep (100U/mL). BR5 mouse cell lines were grown in DMEM (CORNING cellgro, 15-017-CV) supplemented with 10% FBS, penicillin and streptomycin (100 U/ml each), and 1% L-glutamine. KB1P mouse breast tumor cells were grown in DMEM/F12 GlutaMAX supplemented with 10% FBS, 1% Pen Strep (100U/mL), 5 ug/mL insulin, 5 ng/mL cholera toxin, and 5 ng/mL murine epidermal growth factor (RGF) under low oxygen conditions (3% O₂ and 5% CO₂). The generation of RPE1-hTERT TP53/BRCA1 KO Cas9 cells were described elsewhere¹⁶ PEO1 and C4-2 cell lines were described elsewhere⁴⁶. The generation of the MDA-MB-436 BRCA1 +/+ cells lines were described elsewhere⁴⁵. Generation of BR5 and BR5-R1 cell lines were described elsewhere⁵³. The generation of KBP1 cell lines were described elsewhere⁵². Cells were validated by western blot and/or Cell-Titer-Glo/Colony survival assays.

Chemicals: The following chemicals were used in this study: PARP inhibitor olaparib (AZD-2281, SelleckChem), DNAPKi (AZD7648, SelleckChem), PFM01 (HY-116770, MedChem Express), Mirin (M9948, Millipore Sigma), Neocarzinostatin (NCS) (N9162, Millipore Sigma), 5-(Hydroxymethyl)-2′-deoxyuridine (hMDU) (23381, Cayman Chemical), and SN-38 (S4908, Selleckchem).

γ-H2AX Immunofluorescence: Cells were seeded onto coverslips in 6-well plates. Cells were put on ice for 1 minute then pre-extracted with ice cold PBS+0.5% Triton on ice for 1 minute. Cells were then fixed with 4% paraformaldehyde in PBS for 10 minutes. Cells were washed twice with PBS-T (0.01% Tween) and incubated with primary antibody (γH2AX: EMD Millipore JBW301 1:500 dilution in PBS+3% BSA) for 1 hour at room temperature. After three PBS-T washes cells were incubated with secondary antibody (Alexa fluor 488: ThermoFisher A32731 in 1:250 dilution in PBS+3% BSA) for 1 hour at room temperature. After three PBS-T washed cells were incubated with Hoechst stain (ThermoFisher 62249:1:500 dilution in PBS) for 30 minutes at room temperature. After two PBS washes coverslips were mounted with Prolong (Invitrogen, P36930). Images were collected by fluorescent microscopy (Axioplan 2 imaging and Axio Observer, Zeiss) at a constant exposure time in each experiment. Representative images were processed by ImageJ software. Mean intensity of immunofluorescence for each nucleus were measured with Cell Profiler software from the Broad Institute. At least three independent experiments are represented with the mean±SEM.

PARPi survival assays: Cells were seeded into a 6-well plate in biological triplicates for each condition (100-1,000 cells per well) and incubated overnight. The following day, cells were treated with increasing doses of PARPi as indicated in corresponding figures and maintained in complete media for 10-14 days. Percent survival was measured by manual cell counting after methanol/0.5% crystal violet staining.

sgRNAs: sgRNAs were ordered through IDT (Alt-R CRISPR-Cas9 sgRNA). sgRNAs targeting 4, 13, 15, and 17 sites were described in³⁵ and targeted the NF2 gene (4 target sites) and the RPL12 pseudogenes (13, 15, and 17 target sites). sgRNA targeting 1 site (AAVS1) and 100 sites (CAG) were generated by the Brodsky Lab. KDR, LACZ, CXOrf66, and GRFAL were chosen based on the following papers as they are nonessential genes (Mair et al., Cell Rep. 2019 Apr. 9; 27(2):599-615.e12; Hart et al., Mol Syst Biol. 2014 Jul. 1; 10(7):733).

| | | SEQ |
|---|---|---|
| | | ID |
| Target | Spacer sequence | NO: |
| AAVS1 | 5- GTCCCCTCCACCCCACAGTG-3 | |
| HS4 | 5-TGGACTGCAGTACACAATCA-3 | |
| HS13 | 5-AGAAAAACATTAAACACAGT-3 | |
| HS15 | 5-TTTTTGGAGACAGACCCAGG-3 | |
| HS17 | 5-CAGACAGGCCCAGATTGAGG-3 | |
| CAG | 5-AGCAGCAGCAGCAGCAGCAGCAG-3 | |
| Non-targeting | 5- GTAGCGAACGTGTCCGGCGT-3 | |
| KDR-1 | 5-GAGGTGACTGAGTGCAGCGA-3 | |
| LacZ | 5-CCCGAATCTCTATCGTGCGG-3 | |
| CX | 5-CACCTGGCTTACCGAACCTA-3 | |
| GFRAL | 5-CGTTCTACTCCTTAAGTAGT-3 | |

Lipofectamine sgRNA Transfection: ThermoFisher Lipofectamine RNAiMax transfection reagent (13778030) was used for transfecting sgRNAs into RPE1 cell lines already containing Cas9. Transfections were performed in 6-well dishes with 400,000 cells seeded in Pen Strep free media. Each reaction contained 25 nM of the appropriate sgRNA. A control was performed that contained all the buffers and reagents except the sgRNA. Cells were incubated at 37 C for 24 hours prior to setting up colony survival assays.

CRISPR Colony sensitivity assays: Cells were seeded into a 6-well plate (cell numbers varied based on cell line and condition) and maintained in complete media for 10-14 days. Percent survival was measured by manual cell counting after methanol/0.5% crystal violet staining. At least 3 independent assays were performed for each cell line. Survival was normalized to the control that did not have the Cas9 or sgRNA added, but was otherwise treated with all the same buffers and conditions.

CRISPR Cell Titer Glo sensitivity assays: Cells were seeded into 96-well plates (400 cells per well) and incubated at 37 C for 5 days. Percent survival was measured photometrically using the CellTiter-Glo 2.0 viability assay (Promega) in a microplate reader (Beckman Coulter DTX880 Multimode Detector). Survival was normalized to the control that did not have Cas9 or sgRNA added, but was otherwise treated with all the same buffers and conditions.

Neon Electroporation: PEO1, C4-2, and MDA-MB-436 cells were electroporated using the Neon transfection system (ThermoFisher) for CRISPR DSB assays. 5 picomoles of 3xNLS-spCas9 (Wolfe Lab) were mixed with 6.25 picomoles of sgRNA in R-buffer (ThermoFisher) and incubated at room temperature for 10 minutes. The Cas9 RNP complex was mixed with 120,000 cells resuspended in R-buffer. 10 μL of the mixture was electroporated using a 10 μL Neon tip (1,200V, 20 ms, 4 pulses) and plated into a 24-well plates with 500 μL of media. A control was performed containing the buffer, cells, and was electroporated, but did not contain the RNP complex. Cells were incubated at 37 C for two days prior to setting up colony sensitivity assays.

Lipofectamine Cas9 Transfection: ThermoFisher Lipofectamine CRISPRMAX Cas9 transfection reagent (CMAX00003) was used for transfecting spCas9 (Wolfe Lab) into RPE1, BR5, and HeLa cell lines and Cas9-D10A/H840A (IDT) into all cell lines. Transfections were performed in 6-well dishes with 400,000 cells seeded in Pen Strep free media. Each reaction contained 6250 ng of Cas9 nuclease and 1200 ng of the appropriate sgRNA. A control was performed that contained all the buffers and reagents except the Cas9 and sgRNA. Cells were incubated at 37 C for 24 hours prior to setting up colony survival assays.

Lipofectamine 3000 Transfection: ThermoFisher Lipofectamine 3000 transfection reagent (L3000008) was used for transfecting sgRNA and plasmid containing Cas9-t7 exonuclease fusion construct (generated by mutating D10A on a plasmid containing Cas9-t7 exonuclease fusion, see, e.g., Zhang et al., Microbial Cell Factories 21:182 (2022)) into RPE1 cell lines. Transfections were performed in 6-well dishes with 400,000 cells seeded in Pen Strep free media. Each reaction contained 1.25 ug of Cas9-exonuclease plasmid and 1.25 ug of the appropriate sgRNA. A control was performed that contained all the buffers and reagents except the Cas9-exonuclease plasmid and sgRNA. Cells were incubated at 37 C for 24 hours prior to setting up colony survival assays.

DNAPK, Rad51, and RPA70, and 53BP1 Immunofluorescence: Cells were seeded onto coverslips in 6-well plates. Cells were put on ice for 1 minute then pre-extracted with ice cold PBS+0.25% Triton on ice for 1 minute and washed with PBS. Cells were then fixed with 4% paraformaldehyde in PBS for 15 minutes. Cells were pre-extracted again with ice cold PBS+0.5% Triton on ice for 1 minute and washed with PBS. 3% BSA in PBS blocking solution was added for 30 minutes at room temperature. Cells were washed twice with PBS-T (0.01% Tween) and incubated with primary antibody (DNAPK: Abcam ab18192 1:200 dilution, Rad51: Bioacademia 70-001 1:100 dilution, and RPA70: Cell signaling 2267 1:50, 53BP1: Novus Biologicals NB100-304 1:500 dilution in PBS+3% BSA) for 1 hour at room temperature. After three PBS-T washes cells were incubated with secondary antibody (Alexa fluor 488: ThermoFisher A32731 in 1:250 dilution in PBS+3% BSA) for 1 hour at room temperature. After three PBS-T washed cells were incubated with Hoechst stain (ThermoFisher 62249:1:500 dilution in PBS) for 30 minutes at room temperature. After two PBS washes coverslips were mounted with Prolong (Invitrogen, P36930). Images were collected by fluorescent microscopy (Axioplan 2 imaging and Axio Observer, Zeiss) at a constant exposure time in each experiment. Number of foci or mean intensity of immunofluorescence for each nucleus were measured with Cell Profiler software from the Broad Institute. At least three independent experiments are represented with the mean±SEM.

ATM/ATR-P Immunofluorescence: Cells were seeded onto coverslips in 6-well plates. Cells were washed once with PBS then fixed with 4% paraformaldehyde in PBS for 15 minutes. Cells were washed once with PBS then permeabilized with ice cold PBS+0.5% Triton on ice for 1 minute and washed with PBS. 3% BSA in PBS blocking solution was added for 1 hour at 4C. Cells were washed three times with PBS and incubated with primary antibody (ATM/ATR-P: Cell signalling #2851 1:500 in PBS+3% BSA) for 1 hour at room temperature. After three PBS washes cells were incubated with secondary antibody (Alexa fluor 488: ThermoFisher A32731 in 1:250 dilution in PBS+3% BSA) for 1 hour at room temperature. After three PBS washes cells were incubated with Hoechst stain (ThermoFisher 62249:1:500 dilution in PBS) for 30 minutes at room temperature. After two PBS washes coverslips were mounted with Prolong (Invitrogen, P36930). Images were collected by fluorescent microscopy (Axioplan 2 imaging and Axio Observer, Zeiss) at a constant exposure time in each experiment. Mean intensity of immunofluorescence for each nucleus were measured with Cell Profiler software from the Broad Institute. At least three independent experiments are represented with the mean±SEM.

Edu Click-It Immunofluorescence: Cells were grown on coverslips in 6-well plates and 24 hours post Cas9 transfection 10 uM EdU was added for 30 minutes at 37C. Cells were put on ice for 1 minute then pre-extracted with ice cold PBS+0.25% Triton on ice for 1 minute and washed with PSB. Cells were then fixed with 4% paraformaldehyde in PBS for 15 minutes. Cells were pre-extraced again with ice cold PBS+0.5% Triton on ice for 1 minute and washed with PBS. 3% BSA in PBS blocking solution was added for 30 minutes at room temperature. Cells were washed twice with PBS-T (0.01% Tween) EdU labeling was performed using Click-iT EdU Alexa Fluor 488 Imaging Kit (Invitrogen, C10337) according to the manufacturer's instructions. After two PBS washed cells were incubated with Hoechst stain (ThermoFisher 62249:1:500 dilution in PBS) for 30 minutes at room temperature. After two PBS washes coverslips were mounted with Prolong (Invitrogen, P36930). Images were collected by fluorescent microscopy (Axioplan 2 imaging and Axio Observer, Zeiss) at a constant exposure time in each experiment. Mean intensity of immunofluorescence for each nucleus were measured with Cell Profiler software from the Broad Institute. At least three independent experiments are represented with the mean±SEM.

Micronuclei detection by Immunofluorescence: Immunofluorescence was performed exactly as described in the g-H2AX immunofluorescence methods. For analysis, cells with nuclear content (stained with Hoechst) outside the main nucleus were counted by hand using ImageJ software as well as the total number of cells. The percentage of total cells for each condition with micronuclei is reported.

Nick Translation: Cells were grown on coverslips in 6-well plates and 24 hours post Cas9-nickase transfection cells were washed with PBS. Cells were put on ice and pre-extracted with ice cold PBS+0.5% Triton on ice for 5 minutes. Cells were washed 3× with PBST (PBS+0.01% Tween-20). 80 uL filling reaction added to cells. The filling reaction contains 8 uL NEB buffer 2, 2.4 uL 10 mM dNTPs (dA, dG, dC, and BrdUPT), 2.4 μL DNA Pol I (24U), and 67.2 μL sterile distilled water. Cells put in a humdufued incubator at 37C and 5% CO2 for 30 minutes. Cells washed 3× with PBST. Cells fixed for 15 minutes at room temperature with 3% PFA with 2% sucrose in PBS as pH7. Cells washed 1× with 100 mM glycine then incubated in 100 mM glycine for 5 minutes at room temperature. Cells washed 3× with PBST. Denaturing with 2.5M HCl for 1 hour at room temperature at room temperature. Cells washed 3× with PBST then blocked with 3% BSA in PBST for 1 hour at room temperature. Cells washed with 3× with PBST then incubated with primary antibody (Rat-anti-BrdU Ab6326 at 1:100 in 3% BSA in PBST) for 1 hour at 37C. Cells washed 3× with PBST then incubated with secondary antibody (Goat-anti-rat AF594 at 1:200 dilution in 3% BSA in PBST) for 1 hour at room temperature. Cells washed 2× with PBST then DNA stained with Hoechst (ThermoFisher 62249:1:500 dilution in PBS) for 30 minutes at room temperature. Coverslips mounted to slides with Prolong (Invitrogen, P36930) and edges sealed. Images were collected by fluorescent microscopy (Axioplan 2 imaging and Axio Observer, Zeiss) at a constant exposure time in each experiment. Mean intensity of immunofluorescence for each nucleus was measured with Cell Profiler software from the Broad Institute. At least three independent experiments are represented with the mean±SEM.

Single stranded DNA (ssDNA) Immunofluorescence: Cells were grown on coverslips in 10 μM BrdU for 24 hour before induction of nicks by CRISPR. 24 hours after induction of nicks the cells were washed with PBS and pre-extracted by 0.5% Triton X-100 made in phosphate-buffered saline (PBS) on ice. Cells were then fixed using 4% formaldehyde for 15 min at room temperature (RT), and then permeabilized by 0.5% Triton X-100 in PBS again. Permeabilized cells were then incubated with primary antibodies against BrdU (Abcam 6326) at 37° C. for 1 h. Cells were washed and incubated with secondary antibodies (Alexa Fluor 594) for 1 hour at room temperature. DNA was stained with Hoechst (ThermoFisher 62249:1:500 dilution in PBS) for 30 minutes at room temperature. Coverslips were mounted to slides with prolong (Invitrogen, P36930) and edges sealed. Images were collected by fluorescent microscopy (Axioplan 2 imaging and Axio Observer, Zeiss) at a constant exposure time in each experiment. Mean intensity of immunofluorescence for each nucleus was measured with Cell Profiler software from the Broad Institute. At least three independent experiments are represented with the mean±SEM.

TUNEL: To assess apoptosis, the DeadEnd™ Fluorometic TUNEL System (Promega G3250) was utilized. Cells were plated onto coverslips in 6-well dishes and nicks were induced as described in “Lipofectamine Cas9 transfection” section. 24, 48, and 72 hours after nick induction the TUNEL system protocol was followed. Briefly, cells were fixed with 4% formaldehyde, permeabilized with 0.2% Triton, labeled with the kit's fluorescein-12-dUTP at 3′-OH DNA ends by terminal deoxynucleotidyl transferase (TdT), and counterstained with Hoechst to visualize the nuclei. Images were collected by fluorescent microscopy (Axioplan 2 imaging and Axio Observer, Zeiss) at a constant exposure time in each experiment. The mean intensity of immunofluorescence for each nucleus was measured with Cell Profiler software from the Broad Institute. At least three independent experiments are represented with the mean±SEM.

Immunoblotting and antibodies: Cells were harvested, lysed in RIPA buffer and processed for western blot analysis as described previously. Proteins were separated using SDS-PAGE and electro-transferred to nitrocellulose membranes. Membranes were blocked in 5% not-fat dry milk (NFDM) phosphate-buffered saline (PBS)/Tween 20 and incubated with primary antibodies for overnight at 4° C. Antibodies for western blot analysis included anti-β-actin (Sigma A5441), anti-CtIP (Santa Cruz. SC-271339), anti-53BP1 (Novus Biological NB100-304), and anti-MYC (Millipore Sigma, 05-724). Membranes were washed, incubated with corresponding horseradish peroxidase-linked secondary antibodies (Amersham, GE Healthcare) for 1 h at room temperature (RT) and detected by chemiluminescence imaging system (Bio-Rad).

RNA interference: RPE1 cells were reverse transfected using Lipofectamine RNAiMAX (Invitrogen 13778075) with siRNA targeting CtIP (Dharmacon D-011376-01-0005 and D-011376-02-0005) or a control. Stably transduced cells were generated by infection with pLKO.1 vectors containing shRNAs against non-silencing control (NSC) or one of the shRNAs against CtIP (Dharmacon):

CtiP-3:
5′-AAACCCAGGGCTGCCTTGGAAAAG-3′
CtIP-4:
5′-AAACCCAGGGCTGCCTTGGAAAAG-3′

The information was obtained from Dharmacon website (horizondiscovery.com), and the shRNAs were obtained from the University of Massachusetts Medical School (UMMS) shRNA core facility. Cells were selected by puromycin for 3-5 days before experiments were carried out.

Tumor formation in mice: MDA-MB-436 RR1 cells were either induced with 0 or 100 nick target sites as described in “Lipofectamine Cas9 transfection” section. NOD scid mice (strain: NOD.Cg-Prkdc<scid>/J, stock #: 1303) were purchased from Jackson Laboratory. 25 hours after transfection, 12 five-six-week-old female mice were injected unilaterally with 1×10⁶ cells in 35 μL of 50:50 Matrigel/Collagen I into mammary fat pad one group of six mice received cells that had no nick target sites induced, while the other six mice received cells with 100 nick target sites induced. Tumor onset was determined by palpation of the injection site. Tumor growth was measured twice a week with calipers using the formula (length×width²)/2. The in-vivo experiments were all conducted according to the UMass Chan IACUC regulation and guidelines.

### Example 1. BRCA-Deficient Cells are not Selectively Sensitive to CAS9-Induced DSBs

A key question that does not appear to have been formally tested is whether a DSB or some number of DSBs is more sensitizing to BRCA-deficient cells as compared to BRCA-proficient cells. Here, we addressed that question by employing clustered regularity interspaced palindromic repeat (CRISPR)-Cas9 technology. Cas9 was previously stably incorporated into retinol pigment epithelial (RPE1) cells in which p53 was deleted in BRCA1-proficient or BRCA1 knockout (KO) background to support the viability of BRCA1 deficiency in this non-transformed cell system³⁴. Twenty-four hours after guide RNA (sgRNA) introduction, we verified that DSBs were present by immunofluorescence (IF) detection of g-H2AX foci (FIG. 1A), that was dependent on the number of sgRNA sites in the genome³⁵ (FIG. 1B-C). Both cell lines also showed reduced DNA replication consistent with the Cas9-induced DSBs being sufficient to elicit a replication stress response (FIG. 1D). BRCA1 bolsters HR by driving DNA end resection, generating single-stranded DNA (ssDNA) necessary for BRCA2 to load the recombinase, RAD51^{36,37}. Accordingly, DSBs induced more RAD51 foci in wild-type (WT) cells that in the BRCA1-deficient cells (FIG. 1E, 5B). Also, as expected, micronuclei (MN) were higher and g-H2AX persisted in BRCA1 KO as compared to the WT RPE1 cells (FIG. 5B-C) 38. While the intensity of chromatin bound (CB)-DNAPKcs phosphorylation was elevated in both cell lines (FIG. 1F, 5D), there was an overall greater level in BRCA1-deficient cells perhaps reflecting its elevated activity in the absence of HR30.

To assess the impact of the Cas9-induced DSBs on cell fitness, cells transfected with sgRNAs were plated and colony formation assays were performed. As a control, colony formation was also analyzed in response to the PARP1 inhibitor (PARPi) Olaparib. As expected PARPi reduced cell survival of the BRCA1 KO RPE1 cells (FIG. 5E). Notably, we observed that the WT and BRCA1-deficient RPE1 cells were similarly sensitive to DSB induction with a significant loss of viability at 13 DSB target sites (FIG. 1G). However, we did not observe any significant difference between BRCA-proficient and -deficient cells in either colony formation or shorter-term survival assays (FIG. 1G, 5F). Non-essential genes were targeted as to not confound viability. An RPE1 WT and BRCA1 KO p53 null cell system without stable Cas939 verified that a catalytically active Cas9 as well as sgRNAs with genomic targets were required to induce MN as well as cell toxicity. The results were similar between BRCA1-proficient or -deficient cells, whereas as expected, BRCA1 KO cells were sensitive to PARPi (FIG. 5G-L). While BRCA1 status did not impact cell viability, inhibition of DNAPK generated extreme sensitivity (FIG. 1H) consistent with NHEJ being a major repair pathway for blunt ended DSBs especially in the absence of a donor template⁴⁰.

### Example 2. BRCA-Deficient Cells are Selectively Sensitive to Cas9-Induced Nicks

The lesion more likely to sensitize BRCA-deficient cells may be a one-ended DSB that forms when DNA replication collapses at a nick⁴¹. To test this possibility, we employed the same sgRNAs combined with either Cas9 mutant D10A or H840A nickases that generate nicks on opposite strands, the target strand of DNA and non-target strand of DNA, respectively^{42,43} (FIG. 2A). For simplicity, unless noted in the legend and axis, D10A was used for the experiment. Twenty-four hours after sgRNA introduction, we verified that nicks were present by the induction of nick end labelling using nick translation⁴⁴ that appeared to saturate around ~13 nick target sites (FIG. 2B-C, 6A). We further observed that unlike Cas9-induced DSBs, nicks did not impair replication, increase MN formation, or sensitize cells to DNAPK inhibition (FIG. 2D-E, 6B-C). However, in contrast to DSBs, we observed BRCA1 KO cells were significantly more sensitive to nicks compared to WT cells (FIG. 2F, 6D) demonstrating that nicks may induce a toxicity mechanism distinct from DSBs. This nick sensitivity extended to BRCA-deficient cancer cells, including MDA-MB-436 breast cancer cells with vector as compared to BRCA1-complemented⁴⁵, BRCA2 mutant PEO1 as compared to revertant C4-2 ovarian cancer cells⁴⁶, and BRCA2 KO as compared to WT HeLa endometrial cancer cells⁴⁷ that otherwise had the expected PARPi sensitivity (FIG. 2G-I, 6E-I). Moreover, irrespective of BRCA status, the cells displayed a similar sensitivity profile to DSBs with significant fitness loss at 13 DSB target sites (FIG. 2J-L). Collectively, these data suggest that nicks selectively kill BRCA-deficient cells not BRCA-proficient cells.

### Example 3. Nick Resection Generates a Hyper-Nick Sensitivity

If nicks ultimately convert to DSBs, the nick sensitivity should be suppressed by restoration of HR. HR is restored in BRCA1-deficient cells by the loss of the anti-resection factor, 53BP1^{33,48}. As observed with the other RPE1 cell lines, we observed that the WT and BRCA1 53BP1 KO (DKO) cells displayed similar sensitivity profiles to PARPi and Cas9-induced DSBs (FIGS. 7A-B) and that nick induction did not measurably alter the level of EdU incorporation (FIG. 7C). However, the DKO cells had greater sensitivity to nicks than either the WT or BRCA1 KO cells (FIG. 3A), suggesting restored HR in the DKO cells did not provide protection. Moreover, we observed that single 53BP1-KO cells also had greater sensitivity to nicks compared to WT cells (FIG. 7D), further suggesting that 53BP1 is critical for protecting from nick induced sensitivity. 53BP1 is part of a Shieldin complex that “shields” DSBs from end resection and counters HR in BRCA1-deficient cells¹²⁻¹⁶. Thus, to verify the relationship between resection and nick sensitivity, we employed BRCA1 KO cells with deletion of Shieldin factors SHLD3, REV7, or SHLD2. Following nickase introduction and colony formation analysis, we observed a nick sensitivity that was similar to BRCA1 53BP1 DKO cells (FIG. 3B-D).

Notably, colony survival indicated that sensitivity was only marginally elevated with increasing number of nicks (FIG. 3A-D). This finding was mirrored by cell death analysis by terminal deoxynucleotidyl transferase biotin-dUTP nick-end labeling (TUNEL), a method for detecting apoptotic DNA fragmentation. We observed that one nick target site was as efficient as 100 nick target sites inducing cell death in the DKO cells (FIG. 3E). A comparable level of sensitivity to nicks was observed in DKO cells when other genomic regions were targeted with a single nick (FIG. 7F), suggesting that nick sensitivity may not be heavily influenced by the genomic context.

Conceivably, one nick could be as toxic as several nicks, if a single nick undergoes a large ssDNA expansion. To test this idea, we analyzed ssDNA formation in response to nick induction by IF analysis of the ssDNA binding protein RPA and the phosphorylation of substrates following the ssDNA activation of ATR/ATM kinases. We observed an RPA signal in the WT and DKO cells following nicks at ~100 target sites (FIG. 7G). Moreover, DKO cells were positive for ATR/ATM signal along with a non-denaturing IF signal consistent with ssDNA detection with even nicks induced at one target sites (FIG. 3F-G). While less pronounced, similar trends were observed in the BRCA1 KO cells (Figure SH-J). Together, these findings suggest that a single nick in the DKO cells is capable of hyper-resection, checkpoint activation, and cell death.

To directly address if nick resection confers nick sensitivity, we depleted CtIP, a nuclease that mediates resection in the BRCA1 53BP1 DKO cells^{49,50}. With either of two unique shRNA reagents, we observed a reduction in CtIP expression and nick sensitivity within these cells (FIG. 3H-I). We also observed reduced ssDNA after induction of nicks at 100 target sites with reduced CtIP expression consistent with a reduction in resection (FIG. 8A) This reduction in nick-induced sensitivity upon CtIP depletion was also evident when using two separate siRNAs targeting CtIP (FIG. 8B-C). By comparison, replacing the nickase with a nickase-T7 exonuclease fusion in which nick induction is directly linked to resection, CtIP depletion did not elevate cell survival, consistent with resection of a nick being toxic (FIG. 3J). Unlike CtiP loss, MRE11 inhibition did not restore resistance to nick induced sensitivity (FIG. 4D-E), suggesting that CtiP functions distinctly.

The ssDNA binding protein, SHLD2 is the key downstream mediator of 53BP1 that limits DSB resection¹⁶. Accordingly, introduction of SHLD2-fused RNF8 FHA domain, which localizes Shieldin to DSBs independent of 53BP1, suppressed RAD51 foci formation in BRCA1 53BP1 DKO cells¹⁶. Notably, we observed that introduction of the SHLD2-fused RNF8 FHA domain, which allows for forced recruitment of SHLD2 to chromatin independent of 53BP1, suppressed nick sensitivity similar to introduction of WT 53BP1 (FIGS. 3K, 7F-H). Thus, a key effector of 53BP1 anti-resection activity at DSBs or nicks is SHLD2¹⁶. Together, these findings suggest that nick resection underlies the cell killing.

### Example 4. Tumors Targetable with Nicks

Tumors with BRCA1 mutations and low levels of the 53BP1-Shieldin complex, especially some breast and ovarian cancer, should be amenable to nicks as a treatment option⁵¹. To address this possibility, we employed a BRCA1-mutated mouse mammary model (KB1P) in which 53BP1 loss confers PARPi resistance⁵². Notably, the KBIP-177.a5 (BRCA1 53BP1 KO) cells displayed sensitivity to nicks that was greater than the single KBP1-G3 (BRCA1 KO) cell line (FIG. 4A-B). Similarly, the BRCA1 mutant MDA-MB-436 breast cancer cell line that is PARPi resistant and maintains reduced 53BP1 expression (RR1)⁴⁵ displayed sensitivity to nicks greater than the BRCA1-complemented cell line (FIG. 4C-D, 9A-B). As shown with the RPE1 DKO cell line, the MDA-MB-436 RR1 resistant cell line also showed extensive resection with one nick target sites (FIG. 9C) providing support that elevated resection is an opportunity for selective sensitization. By contrast, the BRCA1-deficient mouse ovarian tumor cell line, BR5, is sensitive to nicks, while its PARPi resistant derived line BR5-R1⁵³ displayed nick resistance (FIG. 9D-E) demonstrating that not all PARPi resistant cells are nick sensitive. Indeed, unlike the PARPi resistant RPE1 DKO or MDA-MB-436 RR1, the BR5-R1 resistant cell line does not show a hyper-resection phenotype after nick induction compared to the BR5 cells (FIG. 9F).

We next examined whether the nick vulnerability exposed by 53BP1 loss is exploitable in vivo by analyzing tumor formation in the mammary fat pad of female mice⁵⁴. We divided twelve mice into two groups and administered one million MDA-MB-436 RR-1 cells that underwent transfection procedures with either no nicks vs treated with Cas9-nickase (D10A) and sgRNAs with 100 nick target sites (FIG. 4E). We closely monitored the subsequent tumor formation and growth. After 44 days, the group without induced nicks showed an average tumor size of 1,058 mm³. Interestingly, the group with nicks at 100 target sites exhibited delayed tumor formation and resulted significantly smaller tumors at day 44 with an average tumor size of 172 mm³ (FIG. 4F, 9G). Collectively, these findings suggest that PARPi resistant cells with gains in resection are potentially amenable to nick inducing therapies. Consistent with this interpretation, the DKO cells also displayed sensitivity to genotoxic agents, NCS, hMDU, and SN-38 that generate a large proportion of SSBs compared to other lesions (FIG. 9H-J)^{55,56}.

### REFERENCES

1 Hanahan, D. & Weinberg, R. A. Hallmarks of cancer: the next generation. Cell 144, 646-674, doi:10.1016/j.cell.2011.02.013 (2011).
2 Jackson, S. P. & Bartek, J. The DNA-damage response in human biology and disease. Nature 461, 1071-1078, doi:10.1038/nature08467 (2009).
3 Smeenk, G. & van Attikum, H. The chromatin response to DNA breaks: leaving a mark on genome integrity. Annu Rev Biochem 82, 55-80, doi:10.1146/annurev-biochem-061809-174504 (2013).
4 Futreal, P. A. et al. BRCA1 mutations in primary breast and ovarian carcinomas. Science 266, 120-122, doi:10.1126/science.7939630 (1994).
5 Miki, Y. et al. A strong candidate for the breast and ovarian cancer susceptibility gene BRCA1. δcience 266, 66-71, doi:10.1126/science.7545954 (1994).
6 Godwin, A. K. et al. A common region of deletion on chromosome 17q in both sporadic and familial epithelial ovarian tumors distal to BRCA1. Am J Hum Genet 55, 666-677 (1994).
7 Narod, S. A. & Foulkes, W. D. BRCA1 and BRCA2: 1994 and beyond. Nat Rev Cancer 4, 665-676, doi:10.1038/nrc1431 (2004).
8 Silver, D. P. & Livingston, D. M. Mechanisms of BRCA1 tumor suppression. Cancer Discov 2, 679-684, doi:10.1158/2159-8290.CD-12-0221 (2012).
9 Bryant, H. E. et al. Specific killing of BRCA2-deficient tumours with inhibitors of poly(ADP-ribose) polymerase. Nature 434, 913-917 (2005).
10 Farmer, H. et al. Targeting the DNA repair defect in BRCA mutant cells as a therapeutic strategy. Nature 434, 917-921 (2005).
11 Pilie, P. G., Gay, C. M., Byers, L. A., O'Connor, M. J. & Yap, T. A. PARP Inhibitors: Extending Benefit Beyond BRCA-Mutant Cancers. Clin Cancer Res 25, 3759-3771, doi:10.1158/1078-0432.CCR-18-0968 (2019).
12 Dev, H. et al. Shieldin complex promotes DNA end-joining and counters homologous recombination in BRCA1-null cells. Nat Cell Biol 20, 954-965, doi:10.1038/s41556-018-0140-1 (2018).
13 Ghezraoui, H. et al. 53BP1 cooperation with the REV7-shieldin complex underpins DNA structure-specific NHEJ. Nature 560, 122-127, doi:10.1038/s41586-018-0362-1 (2018).
14 Gupta, R. et al. DNA Repair Network Analysis Reveals Shieldin as a Key Regulator of NHEJ and PARP Inhibitor Sensitivity. Cell 173, 972-988 e923, doi:10.1016/j.cell.2018.03.050 (2018).
15 Mirman, Z. et al. 53BP1-RIF1-shieldin counteracts DSB resection through CST- and Polalpha-dependent fill-in. Nature 560, 112-116, doi:10.1038/s41586-018-0324-7 (2018).
16 Noordermeer, S. M. et al. The shieldin complex mediates 53BP1-dependent DNA repair. Nature 560, 117-121, doi:10.1038/s41586-018-0340-7 (2018).
17 Gao, S. et al. An OB-fold complex controls the repair pathways for DNA double-strand breaks. Nature communications 9, 3925, doi:10.1038/s41467-018-06407-7 (2018).
18 Cong, K. et al. Replication gaps are a key determinant of PARP inhibitor synthetic lethality with BRCA deficiency. Mol Cell, doi:10.1016/j.molcel.2021.06.011 (2021).
19 Hanzlikova, H. et al. The Importance of Poly (ADP-Ribose) Polymerase as a Sensor of Unligated Okazaki Fragments during DNA Replication. Mol Cell 71, 319-331 e313, doi:10.1016/j.molcel.2018.06.004 (2018).
20 Vaitsiankova, A. et al. PARP inhibition impedes the maturation of nascent DNA strands during DNA replication. Nat Struct Mol Biol 29, 329-338, doi:10.1038/s41594-022-00747-1 (2022).
21 Cong, K. & Cantor, S. B. Exploiting replication gaps for cancer therapy. Mol Cell 82, 2363-2369, doi:10.1016/j.molcel.2022.04.023 (2022).
22 Hale, A., Dhoonmoon, A., Straka, J., Nicolae, C. M. & Moldovan, G. L. Multi-step processing of replication stress-derived nascent strand DNA gaps by MRE11 and EXO1 nucleases. Nature communications 14, 6265, doi:10.1038/s41467-023-42011-0 (2023).
23 Simoneau, A., Xiong, R. & Zou, L. The trans cell cycle effects of PARP inhibitors underlie their selectivity toward BRCA1/2-deficient cells. Genes Dev 35, 1271-1289, doi:10.1101/gad.348479.121 (2021).
24 Pommier, Y., O'Connor, M. J. & de Bono, J. Laying a trap to kill cancer cells: PARP inhibitors and their mechanisms of action. Sci Transl Med 8, 362ps317, doi:10.1126/scitranslmed.aaf9246 (2016).
25 Feng, Y. L. et al. DNA nicks induce mutational signatures associated with BRCA1 deficiency. Nature communications 13, 4285, doi:10.1038/s41467-022-32011-x (2022).
26 Jakobsen, K. P. et al. Minimal Resection Takes Place during Break-Induced Replication Repair of Collapsed Replication Forks and Is Controlled by Strand Invasion. Cell Rep 26, 836-844 e833, doi:10.1016/j.celrep.2018.12.108 (2019).
27 Nielsen, I. et al. A Flp-nick system to study repair of a single protein-bound nick in vivo. Nat Methods 6, 753-757, doi:10.1038/nmeth. 1372 (2009).
28 Patel, A. G., Sarkaria, J. N. & Kaufmann, S. H. Nonhomologous end joining drives poly (ADP-ribose) polymerase (PARP) inhibitor lethality in homologous recombination-deficient cells. Proc Natl Acad Sci USA 108, 3406-3411, doi:10.1073/pnas. 1013715108 (2011).
29 Pace, P. et al. Ku70 corrupts DNA repair in the absence of the Fanconi anemia pathway. Science 329, 219-223, doi:10.1126/science.1192277 (2010).
30 Adamo, A. et al. Preventing nonhomologous end joining suppresses DNA repair defects of Fanconi anemia. Molecular cell 39, 25-35, doi:10.1016/j.molcel.2010.06.026 (2010).
31 Clairmont, C. S. et al. TRIP13 regulates DNA repair pathway choice through REV7 conformational change. Nat Cell Biol 22, 87-96, doi:10.1038/s41556-019-0442-y (2020).
32 Li, F. et al. CHAMP1 binds to REV7/FANCV and promotes homologous recombination repair. Cell Rep 40, 111297, doi:10.1016/j.celrep.2022.111297 (2022).
33 Bouwman, P. et al. 53BP1 loss rescues BRCA1 deficiency and is associated with triple-negative and BRCA-mutated breast cancers. Nature structural & molecular biology 17, 688-695, doi:10.1038/nsmb.1831 (2010).
34 Zimmermann, M. et al. CRISPR screens identify genomic ribonucleotides as a source of PARP-trapping lesions. Nature 559, 285-289, doi:10.1038/s41586-018-0291-z (2018).
35 van den Berg, J. et al. A limited number of double-strand DNA breaks is sufficient to delay cell cycle progression. Nucleic Acids Res 46, 10132-10144, doi:10.1093/nar/gky786 (2018).
36 Hustedt, N. & Durocher, D. The control of DNA repair by the cell cycle. Nat Cell Biol 19, 1-9, doi:10.1038/ncb3452 (2016).
37 Tarsounas, M. & Sung, P. The antitumorigenic roles of BRCA1-BARDI in DNA repair and replication. Nat Rev Mol Cell Biol 21, 284-299, doi:10.1038/s41580-020-0218-z (2020).
38 Rothfuss, A. et al. Induced micronucleus frequencies in peripheral lymphocytes as a screening test for carriers of a BRCA1 mutation in breast cancer families. Cancer Res 60, 390-394 (2000).
39 Lim, K. S. et al. USPI Is Required for Replication Fork Protection in BRCA1-Deficient Tumors. Mol Cell 72, 925-941 e924, doi:10.1016/j.molcel.2018.10.045 (2018).
40 Du, J. et al. Quantitative assessment of HR and NHEJ activities via CRISPR/Cas9-induced oligodeoxynucleotide-mediated DSB repair. DNA Repair (Amst) 70, 67-71, doi:10.1016/j.dnarep.2018.09.002 (2018).
41 Serrano-Benitez, A. et al. Unrepaired base excision repair intermediates in template DNA strands trigger replication fork collapse and PARP inhibitor sensitivity. EMBO J 42, e113190, doi:10.15252/embj.2022113190 (2023).
42 Jinek, M. et al. A programmable dual-RNA-guided DNA endonuclease in adaptive bacterial immunity. Science 337, 816-821, doi:10.1126/science.1225829 (2012).
43 Cong, L. et al. Multiplex genome engineering using CRISPR/Cas systems. Science 339, 819-823, doi:10.1126/science.1231143 (2013).
44 Mathew, C. G. Radiolabeling of DNA by nick translation. Methods Mol Biol 2, 257-261, doi:10.1385/0-89603-064-4:257 (1985).
45 Nacson, J. et al. BRCA1 Mutation-Specific Responses to 53BP1 Loss-Induced Homologous Recombination and PARP Inhibitor Resistance. Cell Rep 24, 3513-3527 e3517, doi:10.1016/j.celrep.2018.08.086 (2018).
46 Sakai, W. et al. Functional restoration of BRCA2 protein by secondary BRCA2 mutations in BRCA2-mutated ovarian carcinoma. Cancer Res 69, 6381-6386, doi:0008-5472.CAN-09-1178 [pii] 10.1158/0008-5472.CAN-09-1178 (2009).
47 Thakar, T. et al. Lagging strand gap suppression connects BRCA-mediated fork protection to nucleosome assembly through PCNA-dependent CAF-1 recycling. Nature communications 13, 5323, doi:10.1038/s41467-022-33028-y (2022).
48 Bunting, S. F. et al. 53BP1 inhibits homologous recombination in Brcal-deficient cells by blocking resection of DNA breaks. Cell 141, 243-254, doi:10.1016/j.cell.2010.03.012 (2010).
49 Polato, F. et al. CtIP-mediated resection is essential for viability and can operate independently of BRCA1. J Exp Med 211, 1027-1036, doi:10.1084/jem.20131939 (2014).
50 Sartori, A. A. et al. Human CtIP promotes DNA end resection. Nature 450, 509-514, doi: nature06337 [pii] 10.1038/nature06337 (2007).
51 Clairmont, C. S. & D'Andrea, A. D. REV7 directs DNA repair pathway choice. Trends Cell Biol 31, 965-978, doi:10.1016/j.tcb.2021.05.009 (2021).
52 Jaspers, J. E. et al. Loss of 53BP1 causes PARP inhibitor resistance in Brcal-mutated mouse mammary tumors. Cancer Discov 3, 68-81, doi:10.1158/2159-8290.CD-12-0049 (2013).
53 Yazinski, S. A. et al. ATR inhibition disrupts rewired homologous recombination and fork protection pathways in PARP inhibitor-resistant BRCA-deficient cancer cells. Genes Dev 31, 318-332, doi:10.1101/gad.290957.116 (2017).
54 Torns, E. et al. A new mouse model for the study of human breast cancer metastasis. PLoS One 7, e47995, doi:10.1371/journal.pone.0047995 (2012).
55 Fugger, K. et al. Targeting the nucleotide salvage factor DNPH1 sensitizes BRCA-deficient cells to PARP inhibitors. Science 372, 156-165, doi:10.1126/science.abb4542 (2021).
56 Chen, D. et al. BRCA1 deficiency specific base substitution mutagenesis is dependent on translesion synthesis and regulated by 53BP1. Nature communications 13, 226, doi:10.1038/s41467-021-27872-7 (2022).
57 Zlotorynski, E. Shieldin the ends for 53BP1. Nat Rev Mol Cell Biol 19, 346-347, doi:10.1038/s41580-018-0019-9 (2018).
58 Lukas, C. et al. 53BP1 nuclear bodies form around DNA lesions generated by mitotic transmission of chromosomes under replication stress. Nature cell biology 13, 243-253 (2011).
59 Harrigan, J. A. et al. Replication stress induces 53BP1-containing OPT domains in G1 cells. The Journal of cell biology 193, 97-108 (2011).
60 Audoynaud, C. et al. RNA: DNA hybrids from Okazaki fragments contribute to establish the Ku-mediated barrier to replication-fork degradation. Mol Cell 83, 1061-1074 e1066, doi:10.1016/j.molcel.2023.02.008 (2023).
61 Feng, Z. et al. Rad52 inactivation is synthetically lethal with BRCA2 deficiency. Proc Natl Acad Sci USA 108, 686-691, doi:10.1073/pnas. 1010959107 (2011).
62 Ait Saada, A. et al. Widely spaced and divergent inverted repeats become a potent source of chromosomal rearrangements in long single-stranded DNA regions. Nucleic Acids Res 51, 3722-3734, doi:10.1093/nar/gkad153 (2023).
63 Polleys, E. J., Del Priore, I., Haber, J. E. & Freudenreich, C. H. Structure-forming CAG/CTG repeats interfere with gap repair to cause repeat expansions and chromosome breaks. Nature communications 14, 2469, doi:10.1038/s41467-023-37901-2 (2023).
64 Garcia-Rodriguez, N., Morawska, M., Wong, R. P., Daigaku, Y. & Ulrich, H. D. Spatial separation between replisome- and template-induced replication stress signaling. EMBO J 37, doi:10.15252/embj.201798369 (2018).
65 Mann, A. et al. POLtheta prevents MRE11-NBS1-CtIP-dependent fork breakage in the absence of BRCA2/RAD51 by filling lagging-strand gaps. Mol Cell 82, 4218-4231 e4218, doi:10.1016/j.molcel.2022.09.013 (2022).
66 Schrempf, A. et al. POLtheta processes ssDNA gaps and promotes replication fork progression in BRCA1-deficient cells. Cell Rep 41, 111716, doi:10.1016/j.celrep.2022.111716 (2022).
67 Belan, O. et al. POLQ seals post-replicative ssDNA gaps to maintain genome stability in BRCA-deficient cancer cells. Mol Cell 82, 4664-4680 e4669, doi:10.1016/j.molcel.2022.11.008 (2022).
68 Ceccaldi, R., Rondinelli, B. & D'Andrea, A. D. Repair Pathway Choices and Consequences at the Double-Strand Break. Trends Cell Biol 26, 52-64, doi:10.1016/j.tcb.2015.07.009 (2016).
69 Barazas, M. et al. Radiosensitivity Is an Acquired Vulnerability of PARPi-Resistant BRCA1-Deficient Tumors. Cancer Res 79, 452-460, doi:10.1158/0008-5472.CAN-18-2077 (2019).
70 McGrath, R. A. & Williams, R. W. Reconstruction in vivo of irradiated Escherichia coli deoxyribonucleic acid; the rejoining of broken pieces. Nature 212, 534-535, doi:10.1038/212534a0 (1966).
71 Olive, P. L. The role of DNA single- and double-strand breaks in cell killing by ionizing radiation. Radiat Res 150, S42-51 (1998).
72 Mladenov, E., Magin, S., Soni, A. & Iliakis, G. DNA double-strand break repair as determinant of cellular radiosensitivity to killing and target in radiation therapy. Front Oncol 3, 113, doi:10.3389/fonc.2013.00113 (2013).
73 Zellweger, R. et al. Rad51-mediated replication fork reversal is a global response to genotoxic treatments in human cells. J Cell Biol 208, 563-579, doi:10.1083/jcb.201406099 (2015).
74 Krais, J. J. et al. Genetic separation of Brcal functions reveal mutation-dependent Poltheta vulnerabilities. Nature communications 14, 7714, doi:10.1038/s41467-023-43446-1 (2023).
75 Schochter, F. et al. 53BP1 Accumulation in Circulating Tumor Cells Identifies Chemotherapy-Responsive Metastatic Breast Cancer Patients. Cancers (Basel) 12, doi:10.3390/cancers 12040930 (2020).
76 De Gregoriis, G. et al. DNA repair genes PAXIP1 and TP53BP1 expression is associated with breast cancer prognosis. Cancer Biol Ther 18, 439-449, doi:10.1080/15384047.2017.1323590 (2017).
77 Hurley, R. M. et al. 53BP1 as a potential predictor of response in PARP inhibitor-treated homologous recombination-deficient ovarian cancer. Gynecol Oncol 153, 127-134, doi:10.1016/j.ygyno.2019.01.015 (2019).
78 Eke, I. et al. 53BP1/RIF1 signaling promotes cell survival after multifractionated radiotherapy. Nucleic Acids Res 48, 1314-1326, doi:10.1093/nar/gkz1139 (2020).
79 Huang, A. et al. 53BP1 expression and immunoscore are associated with the efficacy of neoadjuvant chemoradiotherapy for rectal cancer. Strahlenther Onkol 196, 465-473, doi:10.1007/s00066-019-01559-x (2020).
80 Bi, J., Huang, A., Liu, T., Zhang, T. & Ma, H. Expression of DNA damage checkpoint 53BP1 is correlated with prognosis, cell proliferation and apoptosis in colorectal cancer. Int J Clin Exp Pathol 8, 6070-6082 (2015).
81 Yao, J. et al. 53BP1 loss induces chemoresistance of colorectal cancer cells to 5-fluorouracil by inhibiting the ATM-CHK2-P53 pathway. J Cancer Res Clin Oncol 143, 419-431, doi:10.1007/s00432-016-2302-5 (2017).
82 Squatrito, M., Vanoli, F., Schultz, N., Jasin, M. & Holland, E. C. 53BP1 is a haploinsufficient tumor suppressor and protects cells from radiation response in glioma. Cancer Res 72, 5250-5260, doi:10.1158/0008-5472.CAN-12-0045 (2012).
83 Ausborn, N. L. et al. 53BP1 expression is a modifier of the prognostic value of lymph node ratio and CA 19-9 in pancreatic adenocarcinoma. BMC Cancer 13, 155, doi:10.1186/1471-2407-13-155 (2013).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for treating a cancer, the method comprising administering to a cancer cell a CRISPR nickase and at least one single guide RNA (sgRNA) targeting at least one sequence in the cancer cell.

2. The method of claim 1, wherein the sgRNA targets at least two, three, four, five, 10, 15, 20, or 100 sequences in the cancer cell.

3. The method of claim 1, wherein the sgRNA targets AAVS1 or a repeat sequence.

4. The method of claim 1, wherein the CRISPR nickase is a CAS9 nickase.

5. The method of claim 4, wherein the CRISPR nickase is a Cas9 nickase-exonuclease fusion construct.

6. The method of claim 1, wherein the CRISPR nickase and the sgRNA are administered as a nucleic acid.

7. The method of claim 6, wherein the nucleic acid is a viral vector comprising a sequence encoding the CRISPR nickase.

8. The method of claim 1, wherein the CRISPR nickase and the sgRNA are administered as a ribonucleoprotein complex.

9. The method of claim 1, wherein the cancer is a BRCA1-deficient cancer.

10. The method of claim 1, wherein the cancer is a 53BP1-deficient cancer.

11. A method for identifying a subject who has cancer for treatment using the method of claim 1, the method comprising:
contacting cells from the cancer with a CRISPR nickase and a single guide RNA (sgRNA) targeting at least one sequence in the cancer cell, and
determining viability of the cells after contact with the CRISPR nickase and the sgRNA as compared to viability of control cells in the absence of the CRISPR nickase and the sgRNA, wherein a decrease in viability after contact with the CRISPR nickase and the sgRNA as compared to viability in the control cells indicates that the subject is likely to respond to a treatment comprising the method of claim 1.

12. The method of claim 11, further comprising administering the treatment comprising the method of claim 1 to a subject who has a decrease in viability after contact with the CRISPR nickase and the sgRNA.

13. The method of claim 3, wherein the repeat sequence is a CAG repeat.

14. The method of claim 4, wherein the CAS9 nickase is a SpCas9 with a D10A or H840A mutation.

15. The method of claim 5, wherein the exonuclease is a t7 exonuclease.

16. The method of claim 7, wherein the viral vector further comprises a sequence encoding the sgRNA.

17. The method of claim 9, wherein the BRCA1-deficient cancer is breast cancer, triple negative breast cancer, ovarian cancer, fallopian tube cancer, primary cancer, pancreatic cancer, melanoma, or prostate cancer.

18. The method of claim 9, wherein the BRCA1-deficient cancer is a PARPi resistant cancer.

19. The method of claim 10, wherein the 53BP1-deficient cancer is breast cancer, pancreatic cancer, prostate cancer, colorectal cancer, or glioblastoma.

20. The method of claim 10, wherein the 53BP1-deficient cancer is a PARPi resistant cancer.


SEQUENCE LISTING (13 sequences)

SEQ ID NO: 1 - DNA, 20 bp, synthetic construct
  source 1..20: mol_type = other DNA
gtcccctccaccccacagtg

SEQ ID NO: 2 - DNA, 20 bp, synthetic construct
  source 1..20: mol_type = other DNA
tggactgcagtacacaatca

SEQ ID NO: 3 - DNA, 20 bp, synthetic construct
  source 1..20: mol_type = other DNA
agaaaaacattaaacacagt

SEQ ID NO: 4 - DNA, 20 bp, synthetic construct
  source 1..20: mol_type = other DNA
tttttggagacagacccagg

SEQ ID NO: 5 - DNA, 20 bp, synthetic construct
  source 1..20: mol_type = other DNA
cagacaggcccagattgagg

SEQ ID NO: 6 - DNA, 23 bp, synthetic construct
  source 1..23: mol_type = other DNA
agcagcagcagcagcagcagcag

SEQ ID NO: 7 - DNA, 20 bp, synthetic construct
  source 1..20: mol_type = other DNA
gtagcgaacgtgtccggcgt

SEQ ID NO: 8 - DNA, 20 bp, synthetic construct
  source 1..20: mol_type = other DNA
gaggtgactgagtgcagcga

SEQ ID NO: 9 - DNA, 20 bp, synthetic construct
  source 1..20: mol_type = other DNA
cccgaatctctatcgtgcgg

SEQ ID NO: 10 - DNA, 20 bp, synthetic construct
  source 1..20: mol_type = other DNA
cacctggcttaccgaaccta

SEQ ID NO: 11 - DNA, 20 bp, synthetic construct
  source 1..20: mol_type = other DNA
cgttctactccttaagtagt

SEQ ID NO: 12 - DNA, 24 bp, synthetic construct
  source 1..24: mol_type = other DNA
aaacccagggctgccttggaaaag

SEQ ID NO: 13 - DNA, 24 bp, synthetic construct
  source 1..24: mol_type = other DNA
aaacccagggctgccttggaaaag